(19) United States
(12) Patent Application Publication
(10) Pub. No.: US 2011/0003753 A1
(43) Pub. Date: Jan. 6, 2011

(54) COMPOSITIONS AND METHODS FOR DISRUPTING THE FUNCTION OF THE TRANSCRIPTIONAL REPRESSOR COMPONENT Sin3A-PAH2 DOMAIN TO INDUCE DIFFERENTIATION AND GROWTH INHIBITION IN BREAST CANCER

(75) Inventors: SAMUEL WAXMAN, NEW YORK, NY (US); EDUARDO FARIAS, NEW YORK, NY (US)

Correspondence Address: ARENT FOX LLP, 1050 CONNECTICUT AVENUE, N.W., SUITE 400, WASHINGTON, DC, 20036, US

(73) Assignees: SAMUEL WAXMAN CANCER RESEARCH FOUNDATION, NEW YORK, NY (US); C/O SAMUEL WAXMAN CANCER RESEARCH FOUNDATION, NEW YORK, NY (US)

(21) Appl. No.: 12/791,356

(22) Filed: Jun. 1, 2010

Related U.S. Application Data
(60) Provisional application No. 61/182,904, filed on Jun. 1, 2009.

(51) Int. Cl. A61K 38/10; C07K 7/08; C07K 14/435; A61K 38/17; A61P 35/00

(52) U.S. Cl. 514/19.4; 530/327; 530/326; 530/325; 530/324

(57) ABSTRACT

The present invention relates, generally, to compositions for treating breast cancer, use of the compositions to sensitize breast cancer cells to treatment, and methods for treating breast cancer by administering the compositions. The compositions comprise peptides derived from the mSin3A interaction domain (SID) and derivatives thereof, as well as small molecule inhibitors (SMIs) capable of interfering with interactions between SID and the paired amphipathic helix domain 2 (PAH2) domain of the transcription repressor Sin3A. The present invention further relates to compositions for treating triple-negative breast cancer, use of the compositions to render the breast cancer cells susceptible to treatment using hormonal therapies and HER2-based therapies, and methods for treating triple-negative breast cancer by administering the compositions.

### RELATED APPLICATION DATA

This application claims priority to U.S. Provisional Application No. 61/182,904, filed Jun. 1, 2009. The contents of this application are incorporated herein by reference in their entirety.

### BACKGROUND OF THE INVENTION

1. Field of the Invention

The present invention relates, generally, to compositions for treating breast cancer, use of the compositions to sensitize breast cancer cells to treatment, and methods for treating breast cancer by administering the compositions. According to some aspects, the compositions may include peptides derived from the MAD I motif known as the mSin3A interaction domain (SID) and/or small molecule inhibitors (SMIs), where the peptides and/or SMIs are capable of interfering with interactions between SID and the paired amphipathic helix domain 2 (PAH2) domain of the transcription repressor Sin3A. The present invention further relates to compositions for treating triple-negative breast cancer, use of the compositions to render triple-negative breast cancer cells susceptible to treatment using hormonal therapies, HER2-based therapies, and chemotherapies, and methods for treating triple-negative breast cancer by administering the compositions.

2. Description of the Related Art

Following an initial diagnosis of breast cancer, medical professionals select the therapy that is most likely to be effective in treating the cancer by analyzing a number of characteristics of the cancer, such as the size of the tumor(s), the tumor grade, whether the cancer is invasive, involvement of lymph nodes and the number of lymph nodes affected, the hormone receptor status of the tumor(s), the HER2/neu oncogene expression status of the tumor(s), and the margins of resection.

A common way to describe a particular breast cancer is based on the presence, or lack, of three types of hormone receptors that are known to be expressed by most breast cancers: estrogen receptors, progesterone receptors, and human epidermal growth factor receptor 2 (HER2). About 75% of patients with breast cancer have cancer cells that are estrogen-receptor-positive, and about 65% have cells that are both estrogen- and progesterone-receptor-positive. About 20-30% of patients with breast cancer have cancer cells that express too many HER2 receptors.

Unfortunately, about 10-20% of breast cancers are estrogen receptor-negative, progesterone receptor-negative, and HER2-negative. These cancers are known as “triple negative” breast cancer. Depending on the stage of the cancer upon diagnosis, triple negative breast cancer can be extremely aggressive, and more likely to recur and metastasize than other subtypes of breast cancer.

Triple negative breast cancer is typically responsive to chemotherapy, but it can be difficult to treat because it is unresponsive to the most effective receptor-targeted treatments, such as hormonal therapies, including tamoxifen, Arimidex (anastrozole), Aromasin (exemestane), Femara (letrozole), and Faslodex (fulvestrant). Triple-negative breast cancer also does not respond to medications that target HER2, such as Herceptin (trastuzumab) and Tykerb (lapatinib).

In recent years the importance of the role of epigenetic abnormalities in breast cancer, and cancer in general, have come to be better appreciated. Deregulated expression of individual genes arising from aberrant epigenetics frequently involves changes in DNA methylation, and modification of the core histone proteins. Histone deacetylase (HDAC) activities represent one important component of the mechanism(s) underlying gene silencing in cancer. HDACs, however, do not interact directly with chromatin, and are instead recruited via multi subunit co-repressor complexes that bind to transcription factors or other elements of the cellular epigenetic machinery. The HDAC1/2-containing co-repressor complex is the main route by which deacetylation of chromatin-associated histones takes place, and the key adaptor protein in this complex is Sin3. In mammals, there are two highly homologous Sin3 isoforms, Sin3A and B, which were originally identified as MAD binding proteins. Sin3A/B are large, multi-domain proteins that contain four paired amphipathic α-helices known as PAH domains, a central HDAC interaction domain (HID) to which almost all the core co-repressor components bind, and a C-terminal highly conserved region (HCR). As well as serving as a bridge between transcription factors and HDAC activity, the Sin3 complex has also been shown to interact with the methylated DNA-binding protein MeCP2, and the HDAC3-associated co-repressor SMRT. These findings implicate Sin3 proteins in a wide range of chromatin/epigenetic activities.

Accordingly, there is a need in the art for new compositions for treating breast cancer, use of the compositions to sensitize breast cancer cells to treatment, and methods for treating breast cancer by administering the compositions. There is a particular need for compositions, uses of the compositions to enhance the effectiveness of existing treatments, and methods of treatment for triple-negative breast cancer.

### SUMMARY OF THE INVENTION

The present invention relates to compositions for treating breast cancer, use of the compositions to sensitize breast cancer cells to treatment, and methods for treating breast cancer by administering the compositions. The compositions comprise a peptide derived from the mSin3A interaction domain (SID), which will be referred to herein as a SID decoy peptide, and/or small molecule inhibitors (SMIs), where the SID decoy peptides and SMIs are capable of interfering with interactions between SID and the paired amphipathic helix domain 2 (PAH2) domain of the transcription repressor Sin3A. The present invention further relates to compositions for treating triple-negative breast cancer, use of the compositions to render the breast cancer cells susceptible to treatment using hormonal therapies and HER2-based therapies, and methods for treating triple-negative breast cancer by administering the compositions.

Although drugs that inhibit the enzymatic activity of HDACs (HDACi) initially held great promise, a persistent problem has been an inability to develop inhibitors with specificity for individual HDAC isotypes (with the exception of HDAC6). This is also true of drugs that target the activity of DNA methyltransferases (DNMTi), and may have played a part in the limited success achieved thus far with HDACi and DNMTi in clinical applications. Additional problems may be due to cytotoxicity, pleiotropic effects on a wide range of transcription factors, and/or an inability to induce genes required for both growth inhibition and terminal cell division. The strategy with the greatest potential to target aberrant epigenetic states in a specific manner lies with utilizing peptides and small molecules to block specific interactions between oncoproteins and the epigenetic modifiers required for their oncogenic activity. Until “epi-drugs” targeting such enzymatic activities are developed, peptides and small molecule drugs that block specific protein-protein interactions offer another possibility by, for example, targeting the ability of a given oncoprotein to recruit co-repressor complexes to DNA. According to the present invention, Sin3A/B is used as a target for drug development, and to this end the well-characterized PAH2 domain was selected because it binds with high affinity to a small number of SID-containing transcription factors in addition to MAD, particularly for investigation in breast cancer models.

The compositions and methods of the present invention permit existing, highly-effective, hormonal, chemotherapy, and HER2-based treatments for breast cancer to be utilized in cancers that do not express receptors required for those treatments to be effective. There is an unmet need in the art for such compositions, uses, and methods, and particularly for such compositions, uses, and methods directed to the treatment of triple-negative breast cancer.

One aspect of the invention provides an isolated SID decoy peptide derived from the mSin3A interaction domain (SID), wherein said SID decoy peptide blocks interactions between the PAH2 domain of Sin3A and SID-containing proteins, and wherein said SID decoy peptide is from 13 to 20 amino acids long. According to further aspects, the isolated SID peptide comprises VRMNIQMLLEAADYLERRER (SEQ ID No. 1), MNIQMLLEAADYLE (SEQ ID No. 2), MNIQMLLEAPDYLE (SEQ ID No. 3), and/or MNIQMPLEAPDYLE (SEQ ID No. 4).

An additional aspect of the invention provides pharmaceutical dosage form comprising the SID decoy peptide and one or more pharmaceutically acceptable excipients. According to further aspects, the pharmaceutical dosage form of is adapted for administration via a route selected from the group consisting of enteral, parenteral, intravenous, intramuscular, intrathecal, topical, and subcutaneous administration.

According to a further aspect of the invention, uses of the SID decoy peptide are also provided. In some aspects, the uses include 1) inducing chromatin remodeling and re-expression of silenced genes such as E-cadherin, Estrogen Receptor α, and RARβ, so that the cancer cells more closely resemble normal cells; 2) inducing breast cancer morphogenesis, differentiation, adherence, contact inhibition; 3) decreasing invasive colonies, changing colonies from invasive to non-invasive, thereby preventing metastasis; 4) inhibiting tumor growth in vitro and in vivo; 5) increasing sensitivity to estrogen and retinoids in triple-negative breast cancer; 6) activating histone marker H3K4 ME2; 7) decreasing DNA methylation and restoring gene function; 8) compromising the function of JARID; and 9) blocking the function of REST, thereby compromising the function of ZEB1 and allowing re-expression of E-cadherin.

A still further aspect of the invention provides methods of treating breast cancer, comprising administering an effective amount of at least one SID decoy peptide derived from the mSin3A interaction domain (SID), wherein said SID decoy peptide blocks interactions between the PAH2 domain of Sin3A and SID-containing proteins, and wherein said SID decoy peptide is from 13 to 20 amino acids long.

Other novel features and advantages of the present invention will become apparent to those skilled in the art upon examination of the following or upon learning by practice of the invention.

### BRIEF DESCRIPTION OF THE DRAWINGS

FIG. 1: Expression of a decoy peptide corresponding to MAD Sin3 interaction domain (SID) induces markers of differentiation and contact inhibition in MDA-MB-231 breast cancer cells. A. Schematic of the SID peptides and expression constructs used in this study. Shown in the upper panel are the design and sequence of the Tat-SID peptide and Tat-SID^{SCR} scrambled control. The Tat-SID peptide corresponds to amino acids 5-24 of MAD. This sequence binds Sin3 PAH2 with high affinity and has been previously used to study SID-PAH2 interactions in vitro. Peptides contain a leader sequence (YGRKKRRQGGG, SEQ ID No. 5) corresponding to the human immunodeficiency virus type 1 Tat arginine-rich RNA-binding motif (ARM), which has been mutated (RRR>GGG) to improve nuclear entry. B. Morphological changes induced by SID in MDA-MB-231 and MMTV-Myc. The expression of the SID construct induced a cobblestone-like monolayer with well-defined cell-cell contact in contrast to the vector control transfected cells with a spindle shape typical of fully transformed epithelial cells. pSID expresses the minimal MAD SID (amino acids 5-20) with N-terminal SV40 nuclear localization signal (NLS) and a triple FLAG epitope. C. Biochemical analysis showed an increase in the expression of E-cadherin, membrane-associated β-catenin and ZO-1, all of which are involved in cell contact inhibition. Other differentiation-related proteins were also induced, including nuclear-localized RARβ and CRBP1. Consistent with growth arrest was a marked reduction of cyclin D1. D. Confocal IF analyses showing the re-expression of membrane-associated E-cadherin induced by the expression of SID in MDA-MB-231 cells (top row) and MMTV-Myc cells (bottom row) E. Re-expression of nuclear RARβ in MDA-MB-231 cells detected by immunofluorescence microscopy. Scale bars=25 μm.

FIG. 2: SID decoy peptide blocks interactions between Sin3 PAH2 and MAD and interferes with recruitment of members of the Sin3 co-repressor complex. A. GST pull-down of MAD by the Sin3A PAH2 domain is blocked by SID peptide. Sin3A PAH2 domain (amino acids 306-450) was expressed in E. coli as a GST fusion and used in a pull down assay for in vitro interaction with 35S-methionine labeled MAD (left panel) or MAD immunoprecipitated from MDA-MB-231 cell lysates (right panel). 25% of input is shown for in vitro translated MAD and 10% of input for immunoprecipitated MAD (input). Assays were performed using 15 μM Tat-SID or Tat-SID^{SCR} peptide unless otherwise indicated. B. Mammalian two-hybrid analysis shows that SID interferes with recruitment of MAD and HDAC1 by Sin3B. 293T cells were transfected with GAL4^{uas}×5-Tk-Luc reporter together with mammalian two-hybrid vectors expressing a fusion of Sin3B with GAL4 DNA binding domain (GAL4^{DBD}) and fusions of MAD and HDAC1 (columns 5-12 and 14-21, respectively) with VP16 activation domain (VP16^{AD}), as well as SID expression vectors, as indicated. Samples transfected with a vector expressing mutated SID (pSID2^{Mut}), and pTRE and pCMV-3Tag-1A empty vectors were included as negative controls. Luciferase activity was normalized by co-transfection of Renilla luciferase. The parent mammalian two-hybrid vectors, pGALO and pNLVP16, were also used as negative controls. The basal value was set to 1. Values of relative luciferase activity and error bars represent the averages and standard deviations, respectively, of 4 separate experiments. Transfection of the pM3-VP16(AD) positive control vector (Clontech), a fusion of GAL4 DBD and VP16AD resulted in a large (60-fold) increase in relative luciferase activity (not shown). Where indicated, samples were treated with 15 μM Tat-SID or Tat-SID^{SCR} peptide at time of cell plating and after transfection.

FIG. 3: A. Morphogenesis in 3D cultures in Matrigel. MDA-MB-231 and MMTV-Myc cells expressing SID or the double mutant were cultured in 3D-Matrigel for 14 days. Both vector and the double mutant transfected cells developed large and invasive colonies (upper and central panels), meanwhile the cells expressing SID were smaller and non-invasive. Moreover, approximately 25% of the MDA-MB-231 colonies displayed non-polarized rudimental lumens (lower right panel, L indicates lumen). However, at 14 days in Matrigel culture expression patterns of GM130 and caspase-3, a polarization and cavitation markers, respectively, did not indicate full polarization of SID expressing cells. B. Reversion of the invasive phenotype of the MDA-MB-231 cells in Matrigel 3D cultures. Upper panel indicates change in numbers of invasive versus non invasive colonies (as indicated in crosshatched and non-crosshatched bars, respectively) in the presence of different concentrations (as indicated) of cell penetrating SID or SID^{SCR} (scrambled control) peptide. The colonies were counted after 10 days of treatment with a medium containing fresh peptide changed every 24 hours. Lower panel shows phase contrast microscopy indicating the effect of each treatment on colony morphology. The reduction in invasive phenotype was quantified by counting 5 low magnification fields per well done in triplicates (upper panel). The p value was calculated using the unpaired Student t test.

FIG. 4: Expression of the SID domain or the SID-MAD-interacting peptide fuse with GFP impairs tumor growth. A. MMTV-Myc cells (2.5×10⁵ cells/fat pad) were injected into the mammary fat pads of FVB syngeneic mice (n=10). MMTV-Myc cells stably expressing pTHE were injected in the left flank, with vector control injected in the right flank of each mouse as indicated to avoid interanimal variation between groups, and tumors were retrieved after 14 days. B. Immunohistochemical analysis of the tumors indicate that those expressing the SID domain express higher levels of E-cadherin localized to the plasma membrane (upper left panel, insert), higher nuclear expression of p27 and low pRB (8 out of 8 tumors studied). Scale bars=50 μm. Similar results were obtained using pEGFP-N3/SID plasmids.

FIG. 5: A. Schematic representations of the promoters and 5′ untranslated regions (5′UTR) of CDH1 and ESR1 used in this study. Positions of CpG dinucleotides are indicated by grey ticks and the transcription initiation sites are represented by black arrows. The relative positions of primer pairs used to PCR amplify immunoprecipitated DNA from ChIP analysis and bisulfite-modified DNA are also indicated. B. H3K4me3 levels on the CDH1 and ESR1 promoter regions increase dramatically in response to SID. ChIP analysis was performed with chromatin from wild type or stably transfected MDA-MB-231 cells as indicated. Cross-linked protein-DNA complexes were immunoprecipitated with an anti-trimethyl H3K4 antibody and amplified by real-time PCR. Results are shown as percentage of input DNA. The RPL30 housekeeping gene is shown as a control. C. Real-time PCR analysis of CDH1 and ESR1 gene expression. Values are shown as molecules per μg total RNA and were derived from the ΔCt between the GAPDH housekeeping gene and the gene of interest. The amount of GAPDH molecules per μg total RNA was determined by absolute quantification. D. The CDH1 and ESR1 promoters undergo demethylation in response to SID. Following bisulfite modification of wild type or stably transfected MDA-MB-231 cells as indicated, specifically amplified PCR products were sequenced using primers corresponding to the promoter/5′UTR of ESR1. Positions of CpG dinucleotides are indicated by grey ticks and the transcription initiation sites are indicated by black arrows. Five clones were sequenced per sample and a black-filled circle represents where a CpG dinucleotide was found to be methylated. Unmethylated CpGs are by represented open circles. CpG positions are shown relative to transcription initiation site. E. Functional assays for ERα and RAR activation. To determine whether the re-expressed ERα and RARβ were functional in MDA-MB-231 cells transfected with SID (pTHE plasmid), and cell proliferation assays were performed. Cells were stimulated with 2.5 nM estradiol (E2) or E2 plus 2.5 uM tamoxifen overnight (left panel) or 1 mM ATRA (pan-RAR agonist) or 100 nM Am580 (RARα-specific agonist) daily for 48 hrs (right panel) to determine the activation of ERα and RARs respectively.

FIG. 6: H3^{Ac}, H3K4^{me2} and H3K27^{me3} levels on the CDH1 and ESR1 promoter regions increase in response to SID decoy peptide. ChIP analysis was performed with chromatin from MDA-MB-231 cells stably transfected with SID decoy peptide-containing pTHE or pTRE control plasmids, as indicated. Cross-linked protein-DNA complexes were immunoprecipitated with anti-acetyl H3, anti-dimethyl H3K4 or anti-trimethyl H3K27 antibodies as indicated and amplified by real-time PCR. Results are shown as percentage of input DNA.

FIG. 7: A. Transfection of the minimal SID in which the encoded 13 amino acid peptide (SID decoy peptide) was bound to the Sin3A PAH-2 domain and induced phenotypic changes in human and mouse breast cancer cells in vitro and in vivo, where the changes included the induction of differentiated morphogenesis with loss of the invasive colonies on matrigel. B. Matrigel invasion of MDA-MB-231 cells over 24 hours is shown in terms of the number of cells observed in a 20× field. MDA-MB-231 cells transfected with the SID-containing TATS exhibited reduced matrigel invasion.

FIG. 8: Graphs illustrating the increase in E-cadherin gene expression. A. Expression of E-cadherin in estrogen receptor negative breast cancer cell lines MDA-MB-231, MDA-MB-157. B. Expression of E-cadherin in estrogen receptor positive breast cancer cell lines T47D, and MCF7. Levels were determined by qRT-PCR.

FIG. 9: A table showing that the SID decoy peptide induced expression of genes in the human breast cancer cell lines MDA-MB-231, MDA-MB-157, MDA-MB-468, T47D, and MCF7.

FIG. 10: Depiction of the putative order of events in E-cadherin promotion in the presence of a SID decoy peptide.

### DETAILED DESCRIPTION OF THE PRESENTLY PREFERRED EMBODIMENTS

The present invention relates to compositions for treating breast cancer, use of the compositions to sensitize breast cancer cells to treatment, and methods for treating breast cancer by administering the compositions. In some aspects, the compositions comprise compounds capable of interacting with the Sin3A/B protein to disrupt its function, and according to further aspects, the compositions may be derived from the Sin3A interaction domain (SID). According to some aspects, the compositions include peptides derived from the MAD I motif known as the mSin3A interaction domain (SID), which are referred to herein as SID decoy peptides, and/or small molecule inhibitors (SMIs), such as where the SID decoy peptides and SMIs are capable of interfering with interactions between the SID and the paired amphipathic helix domain 2 (PAH2) domain of the transcription repressor Sin3A. The present invention further relates to compositions for treating triple-negative breast cancer, use of the compositions to render the breast cancer cells susceptible to treatment using hormonal therapies, HER2-based therapies, and chemotherapies, and methods for treating triple-negative breast cancer by administering the compositions.

### 1. Compositions

The present invention relates to compositions for treating breast cancer, which may be administered in conjunction with the methods for treating breast cancer. In some aspects, the compositions comprise compounds capable of interacting with the Sin3A/B protein to disrupt its function, and according to further aspects, the compositions are derived from the MAD I motif known as the Sin3A interaction domain (SID). According to some aspects, the compositions include SID decoy peptides derived from the mSin3A interaction domain (SID). According to other aspects, the compositions include small molecule inhibitors (SMIs) that are capable of interfering with interactions between the SID and the paired amphipathic helix domain 2 (PAH2) domain of the transcription repressor Sin3A. According to some aspects, the SMIs may be derived from the SID decoy peptides.

The Sin3A/B-PAH2 domain is a target for disruption because of its role in mediating the effects of a relatively small number of transcription factors containing a PAH2-binding motif known as the Sin3 interaction domain (SID). The crystal structure of the Mad I SID and the mSin3A PAH-2 domain interaction is well defined and can be inhibited by small peptides and/or chemical compounds. According to some aspects of the invention, it has been discovered that in both human and mouse breast cancer cells the targeted disruption of Sin3 function by introduction of a SID decoy peptide interferes with PAH2 binding to SID-containing partner proteins. The development of SID decoy peptides and/or the creation of small molecule inhibitors (SMIs) to block this interaction is therefore one aspect of the present invention. According to further aspects of the invention, these compositions offer another approach for treating cancer, including breast cancer and prostate cancer, and may also have wider therapeutic implications in the treatment of other cancers.

The compositions of the present invention may lead to epigenetic reprogramming and re-expression of breast cancer-associated silenced genes, such as those encoding E-cadherin, estrogen receptor a (ERα), and retinoic acid receptor β (RARβ). The compositions may also lead to impaired tumor growth in vivo. The compositions of the present invention are also effective in the “triple-negative” MDA-MB-231 breast cancer cell line, restoring sensitivity to cancer therapies such as 17β-estradiol, tamoxifen, and retinoids. Sensitivity to other treatments may also be enhanced by the compositions of the invention.

In certain aspects of the invention, SID decoy peptides are provided for use in the treatment of cancer, particularly breast cancer, more particularly advanced breast cancer, and most particularly triple-negative breast cancer. The SID decoy peptides of the present invention induce differentiation and programmed cell death with minimal toxicity to normal cells.

The SID peptide (which comprises at least the amino acid sequence VRMNIQMLLEAADYLERRER, SEQ ID No. 1) may be used as a starting point to prepare the SID decoy peptides of the present invention. According to the compositions of the invention, the SID decoy peptide may comprise the isolated SID peptide, a variant thereof, a fragment thereof, and/or a fusion peptide thereof.

In the context of the instant invention, the terms “oligopeptide”, “polypeptide”, “peptide” and “protein” can be used interchangeably when referring to the SID peptide and SID decoy peptides provided in accordance with the present invention. However, it should be understood that the invention does not relate to the peptides in natural form, i.e., the polypeptides are isolated or obtained by purification from natural sources or obtained from host cells prepared by genetic manipulation (e.g., the peptides, or fragments thereof, are recombinantly produced by host cells, or by chemical synthesis). Peptides according to the instant invention may also contain non-natural amino acids. The terms “oligopeptide”, “polypeptide”, “peptide” and “protein” are also used, in the instant specification, to designate a series of amino acid residues of any length. Linker elements can be joined to the polypeptides of the subject invention through peptide bonds or via chemical bonds. Additionally, the terms “amino acid(s)” and “residue(s)” can be used interchangeably.

A “variant” or “variant peptide” (or “peptide variant”) is a peptide that exhibits certain modifications as compared to the SID peptide. These modifications can include a deletion, addition, or substitution of at least one amino acid (e.g., one, two, three or more amino acids), a truncation, an extension, a chimeric fusion (fusion protein), a mutation, or polypeptides exhibiting post-translational modifications. These modifications can occur anywhere in the peptide, e.g., at one or both ends and/or in the middle. Among the variant peptides included within the scope of the present invention are those comprising amino acid sequences exhibiting between at least (or at least about) 20.00% to 99.99% (inclusive) identity to the full length, native, or naturally occurring polypeptide. The aforementioned range of percent identity is to be taken as including, and providing written description and support for, any fractional percentage, in intervals of 0.01%, between 20.00% and, up to, including 99.99%. These percentages are purely statistical and differences between two polypeptide sequences can be distributed randomly and over the entire sequence length. Thus, variant peptides can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the peptide sequences of the instant invention. In a preferred embodiment, a variant or modified peptide exhibits at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the SID peptide. The percent identity is calculated with reference to the full-length polypeptide or the length of the fragment that is identified.

Functional fragments according to the subject invention can comprise a contiguous span of at least 10 consecutive amino acids of SID, or at least 10 consecutive amino acids of a variant of SID. Peptides fragments according to the present invention can be of any length from at least 10 consecutive amino acids to 1 amino acid less than a full length peptide (e.g., 1 amino acid less than the full length SID peptide). Thus, in some embodiments, functional fragments may be 10, 11, 12, 13, 14, 15, 16, 17, 18, 19, or 20 amino acids in length (e.g., a span of 10, 11, 12, 13, 14, 15, 16, 17, 18, 19, or 20 consecutive amino acids selected from the full length SID peptide). According to some aspects of the invention, the fragments comprise 13 consecutive amino acids. According to other aspects of the invention, the fragments comprise 20 consecutive amino acids.

Each fragment of the subject invention can also be described in terms of its N-terminal and C-terminal positions. For example, combinations of N-terminal to C-terminal fragments of from 10 contiguous amino acids up to 1 amino acid less than the full length peptide of are included in the present invention. Thus, a 10 consecutive amino acid fragment could occupy positions selected from the group consisting of 1-10, 2-11, 3-12, 4-13, 5-14, etc. A 13 consecutive amino acid fragment could occupy positions selected from the group consisting of 5-18, 6-19, 7-20, 8-21, 9-22, etc. A 20 consecutive amino acid fragment could occupy positions selected from the group consisting of 3-22, 4-23, 5-24, 6-25, 7-26, etc.

According to one aspect of the invention, a 20 amino acid peptide based on positions 5 to 24 of the SID peptide is provided (i.e., VRMNIQMLLEAADYLERRER, SEQ ID No. 1). According to another aspect of the invention, a 13 amino acid peptide based on positions 7 to 20 of the SID peptide is provided (i.e., MNIQMLLEAADYLE, SEQ ID No. 2). Mutants of these peptides are also provided, such as mutants of the 13 amino acid peptide, including a single mutant having the sequence MNIQMLLEAPDYLE (SEQ ID No. 3), and a double mutant having the sequence MNIQMPLEAPDYLE (SEQ ID No. 4).

Other proteins having similar domains to the SID peptide may also be used to form the peptides and fragments of the present invention, such as the Sp1/Kruppel-like factors (KLF9/BTEB1, KLF10/TIEG1, KLF11/TIEG2, KLF13/BTEB3, and KLF16/BTEB4), UME6 (a yeast meiosis-specific protein), HBP1 (a negative regulator of several cell cycle-specific and differentiation-specific genes, including those encoding p21/Cip1/Waf1, cyclin D1, MyoD, and N-myc), and REST (a transcriptional silencer).

According to further aspects, the variant peptides and fragments of the present invention retain the ability to interfere with PAH2 binding to SID-containing partner proteins, and also retain the ability to cause epigenetic reprogramming and re-expression of breast cancer-associated silenced genes, such as those encoding E-cadherin, estrogen receptor a (ERα), and retinoic acid receptor β(RARβ). The variant peptides and fragments may also lead to impaired tumor growth in vivo as well as reduced invasiveness. Although these functions are retained by the variant peptides and fragments, the particular mechanism of action by which the function is achieved need not be the same.

According to further aspects of the invention, one or more amino acid residues within the sequence can be substituted by another amino acid of a similar polarity which acts as a functional equivalent, resulting in a silent alteration. In one aspect of the present invention, conservative substitutions for an amino acid within the sequence may be selected from other members of the class to which the amino acid belongs. Conservative substitutions also include substitutions by amino acids having chemically modified side chains that do not eliminate the biological function of the resulting variant.

The peptides of the present invention may be linear or cyclic, and may include (D) as well as (L) amino acids. Peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylamino), with or without any of a wide variety of side chain modifications and/or substitutions (e.g., methylation, benzylation, t-butylation, tosylation, alkoxycarbonylamino, and the liked). Such modifications and derivatives of a peptide sequence, and others known to those of skill in the art, are herein termed “variants”. Preferred derivatives include amino acids having an N-acetyl group (such that the amino group that represents the N-terminus of the linear peptide is acetylated) and/or a C-terminal amide group (i.e., the carboxy terminus of the linear peptide is amidated). Residues other than common amino acids that may be present include, but are not limited to, penicillamine, tetramethylene cysteine, pentamethylene cysteine, mercaptopropionic acid, pentamethylene-mercaptopropionic acid, 2-mercaptobenzene, 2-mercaptoaniline, 2-mercaptoproline, ornithine, diaminobutyric acid, aminoadipic acid, m-aminomethylbenzoic acid, and diaminopropionic acid.

The peptides of the present invention also encompass various peptidomimetics, which are small protein-like chains designed to mimic a peptide. These may be provided either by modifying the peptides of the present invention to form peptoids or β-peptides, and the altered chemical structure of the peptide may provide beneficial molecular properties, such as improved stability or biological activity.

Peptides as described herein may be synthesized by methods well known in the art, including recombinant DNA methods and chemical synthesis. One skilled in the art, using the identified sequences, can synthesize the peptides for use in the invention.

The subject invention also provides polynucleotides comprising nucleotide sequences encoding the peptides of the invention. “Nucleotide sequence”, “polynucleotide” or “nucleic acid” can be used interchangeably, and are understood to mean, according to the present invention, either a double-stranded DNA, a single-stranded DNA or products of transcription of the said DNAs (e.g., RNA molecules). It should also be understood that the present invention does not relate to genomic polynucleotide sequences in their natural environment or natural state.

Polynucleotides can be administered to cells or subjects and expressed by the cells or subjects, rather than administering the peptides themselves. The subject invention also provides genetic constructs comprising a polynucleotide sequence of the invention. Genetic constructs of the subject invention can also contain additional regulatory elements such as promoters and enhancers and, optionally, selectable markers. In one embodiment, host cells that have been genetically modified with a polynucleotide encoding at least one peptide of the invention are administered to a subject to treat a proliferation disorder and/or to reduce the growth of malignant cells. The polynucleotide is expressed by the host cells, thereby producing the peptides within the subject. Preferably, the host cells are allogeneic or autogeneic to the subject.

Also within the scope of the subject instant invention are vectors or expression cassettes containing genetic constructs as set forth herein, or polynucleotides encoding the peptides, operably linked to regulatory elements. The vectors and expression cassettes may contain additional transcriptional control sequences as well. The vectors and expression cassettes may further comprise selectable markers. The expression cassette may contain at least one additional gene, operably linked to control elements, to be co-transformed into the organism. Alternatively, the additional gene(s) and control element(s) can be provided on multiple expression cassettes. Such expression cassettes are provided with a plurality of restriction sites for insertion of the sequences of the invention to be under the transcriptional regulation of the regulatory regions. The expression cassette(s) may additionally contain selectable marker genes operably linked to control elements.

The subject invention also provides for the expression of a peptide, fragment, or variant encoded by a polynucleotide sequence disclosed herein comprising the culture of a host cell transformed with a polynucleotide of the subject invention under conditions that allow for the expression of the peptide and, optionally, recovering the expressed peptide. The invention also encompasses the host cells transformed by a vector according to the invention. These cells may be obtained by introducing into host cells a nucleotide sequence inserted into a vector as defined above, and then culturing the cells under conditions allowing the replication and/or the expression of the polynucleotide sequences of the subject invention.

Dimers, trimers, and other multimers based on the peptides of the present invention are also provided. The multimers of the invention, such as, for example, homodimers or homotrimers, may be formed when peptides of the invention contact one another in solution. In another embodiment, heteromultimers of the invention, such as, for example, heterotrimers or heterotetramers, are formed when peptides of the invention contact antibodies to the peptides of the invention in solution. In other embodiments, multimers of the invention are formed by covalent associations with and/or between the peptides of the invention. Proteins comprising multiple polypeptides of the invention separated by peptide linkers may also be produced using conventional recombinant DNA technology. Multimeric polypeptides can also be generated using chemical techniques known in the art.

Antibodies to the peptide are also provided, which are useful to track the peptides in the body. Antibodies to the peptides have commercial value for use in measuring the concentration of drug in serum, and for other reasons. The antibodies may be generated using existing techniques well-known to those skilled in the art.

According to some aspects of the invention, the peptides and peptidomimetics of the present invention may be used to develop the SMIs of the present invention. According to further aspects of the invention, the antibodies may be used to screen potential candidates for SMIs.

The SID decoy peptides and/or SMIs can be tracked by directly labeling them with a detectable marker such as a radiolabel. By modifying the SID decoy peptides and/or SMIs with radiolabels or other identifiable means, they can be used as agents that could detect breast (or other) cancers earlier than other existing methods. In the case of the SID decoy peptides, the radiolabel may be provided by attaching an additional amino acid to the peptide, the additional amino acid being radiolabeled.

According to some aspects of the invention, the compositions of the present invention may include SID decoy peptides and/or SMIs, additionally combined with any form of hormonal therapy, chemotherapy, or radiation therapy, including, but not limited to tamoxifen, anastrozole, exemestane, letrozole, fulvestrant, trastuzumab, and lapatinib.

### 2. Dosage Forms Containing the Compositions

While SID decoy peptides and/or SMIs of the invention can be administered in vitro and in vivo as isolated agents, it is preferred to administer them as part of a pharmaceutical composition. The present invention therefore provides compositions comprising one or more SID decoy peptides and/or SMIs of the invention in association with at least one pharmaceutically acceptable carrier.

The pharmaceutical compositions can be adapted for various routes of administration, such as enteral, parenteral, intravenous, intramuscular, intrathecal, topical, subcutaneous, and so forth. Administration can be continuous or at distinct intervals, and the most suitable administration route and schedule can be determined by a person of ordinary skill in the art.

Pharmaceutical dosage forms containing the compositions may include at least one isolated peptide of the present invention, or an isolated polynucleotide encoding the isolated peptide; and a pharmaceutically acceptable carrier. In one embodiment, when the polynucleotides of the invention are to be administered, the pharmaceutical composition comprises host cells that have been genetically modified to express the polynucleotide encoding the peptide.

Parenteral dosage forms may be used in certain aspects of the invention to administer the SID decoy peptide compounds and/or SMIs of the present invention, which are capable of interacting with the Sin3A/B protein to disrupt its function. The preferred route of administration of peptides is currently injection, which may be acceptable for treatment of cancer or for early detection of cancer. The parenteral formulations according to the present invention can be prepared in any manner suitable to deliver the SMIs and/or peptides. For example, according to some aspects, the compositions of the present invention can be administered by direct injection into a tumor, or systemically into the circulatory system to kill circulating tumor cells.

Oral and transdermal formulations containing the compositions of the present invention are also envisioned. The oral formulations according to the present invention can be prepared in any manner suitable to deliver the SMIs and/or peptides. Examples of dosage forms for oral administration include a tablet, a caplet, a hard or soft capsule, a lozenge, a cachet, a dispensable powder, granules, a suspension or solution, an elixir, a liquid, or any other form reasonably adapted for oral administration. Transdermal formulations can be prepared in the form of a patch, ointment, cream, lotion, solution, tincture, or the like.

The SMIs and/or peptides can be further formulated together with one or more pharmaceutically acceptable excipients to produce the pharmaceutical compositions of the present invention. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients include, by way of example, buffers, carbohydrates, chelating agents, diluents, disintegrants, binding agents, adhesives, wetting agents, lubricants, glidants, crystallization inhibitors, preservatives, surface modifying agents, flavors, dyes, fragrances, and substances added to improve appearance of the composition.

Excipients employed in compositions of the invention can be solids, semi-solids, liquids or combinations thereof. Compositions of the invention containing excipients can be prepared by any known technique of pharmacy that comprises admixing an excipient with a drug or therapeutic agent.

The SID decoy peptide compounds and/or SMIs of the present invention can be administered to a subject with cancer alone, or they may be co-administered with one or more other therapies, such as hormonal therapy, chemotherapy or radiation therapy. Co-administration can be carried out simultaneously (in the same or separate formulations) or consecutively. According to further aspects of the invention, SID decoy peptide compounds and/or SMIs of the invention can be administered as an adjuvant therapy.

The SID decoy peptide compounds and/or SMIs of the invention, whether administered separately, or as a pharmaceutical composition, can include various other components as additives. The additional components can provide additional therapeutic benefits, act to affect the therapeutic action of the compounds of the invention, or act to preventing any potential side effects which may be posed as a result of administration of the compounds. The SID decoy peptide compounds and/or SMIs of the subject invention can also be conjugated to therapeutic agents in order to provide, for example, targeted delivery of the therapeutic agents.

The pharmaceutical formulations containing SMIs and/or SID decoy peptides can be prepared by any suitable process, not limited to processes described herein.

### 3. Use of the Compositions and Dosage Forms

The present invention also relates to use of SID decoy peptide compounds and/or SMIs, and/or pharmaceutical dosage forms containing them. The SID decoy peptide compounds and/or SMIs, or the dosage forms containing them, are preferably used in order to interfere with interactions between the SID and the paired amphipathic helix domain 2 (PAH2) domain of the transcription repressor Sin3A. According to certain aspects, the compositions and dosage forms are used to interfere with these interactions in cancer cells in vitro or in vivo, and in further aspects they are used to interfere with these interactions in cancer patients in vivo.

In accordance with additional aspects of the invention, the compositions and dosage forms are used to sensitize breast cancer cells to treatment using presently-available therapies, such that the effectiveness of the therapy is increased compared to the level of efficacy of the treatment prior to administration of the compositions and dosage forms. According to certain aspects, the efficacy of the therapy may be increased by about 25%, preferably by about 50%, more preferably by about 60%, and most preferably by about 75%. According to further aspects, the efficacy of the therapy is increased by about 100%, preferably by about 200%, more preferably by about 300%, and most preferably by about 400% or more.

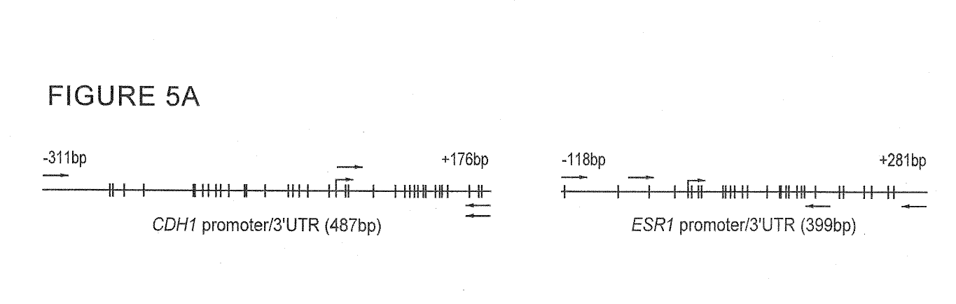

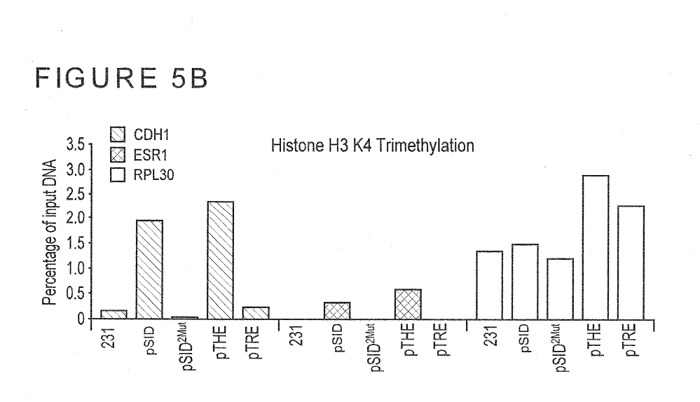

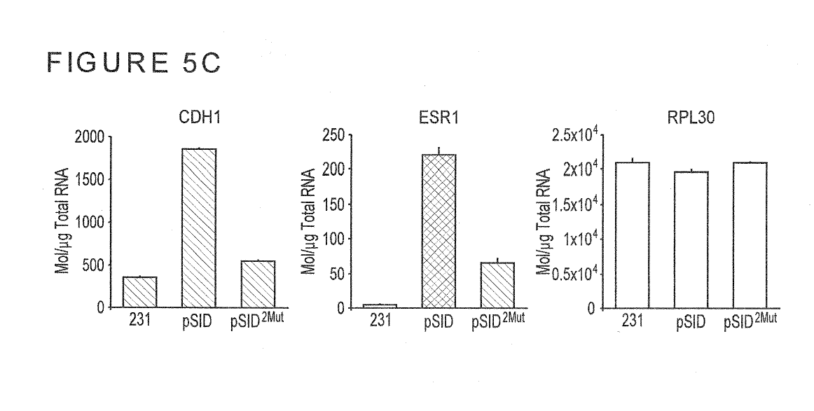

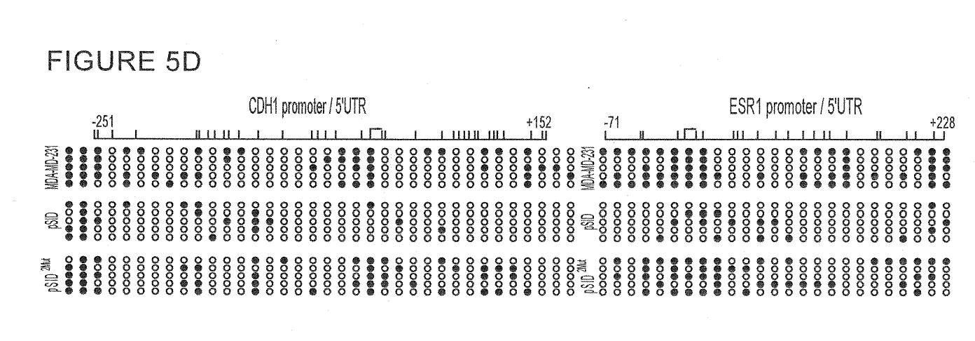

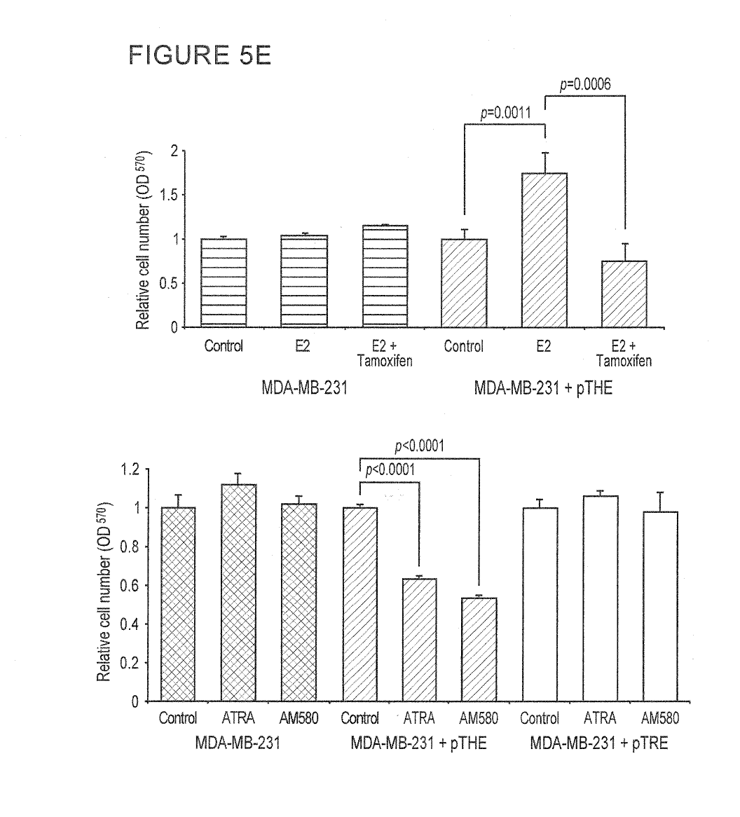

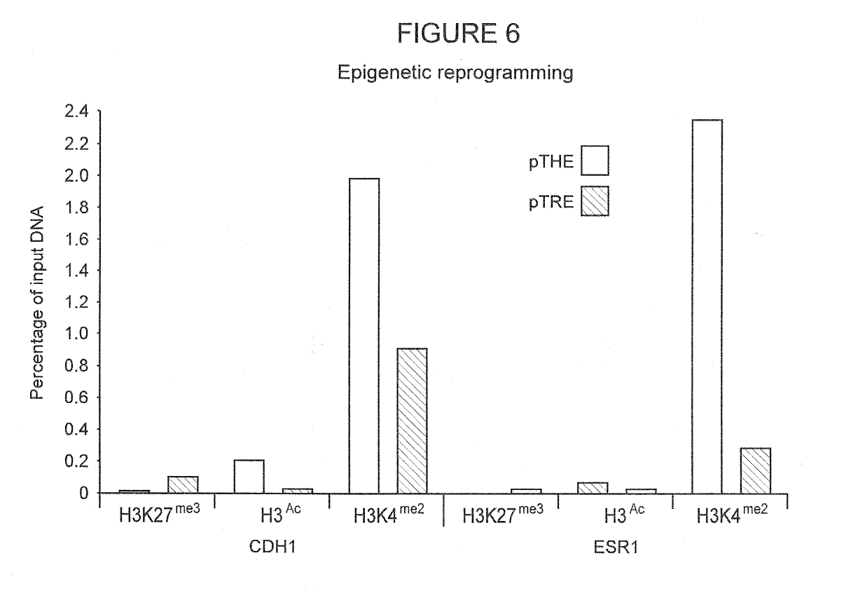

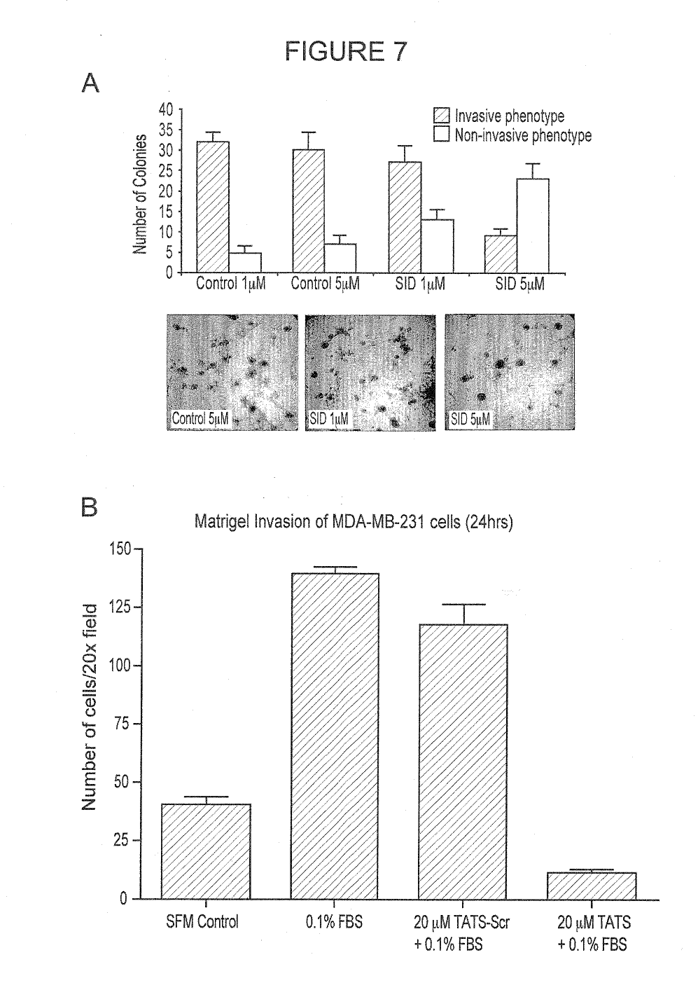

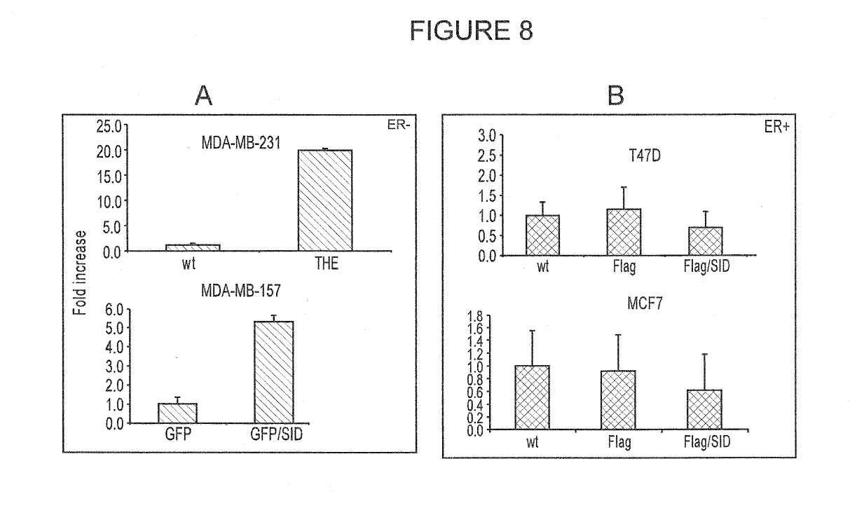

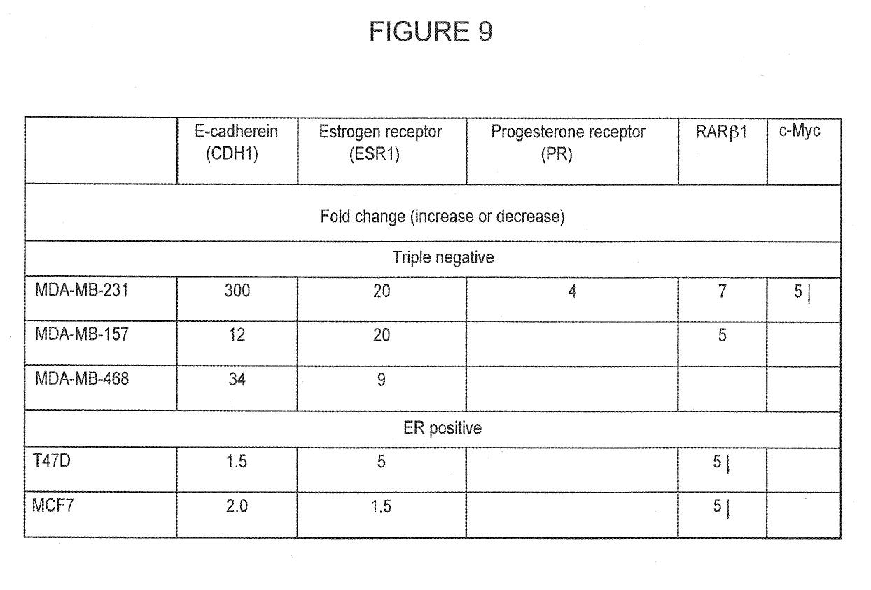

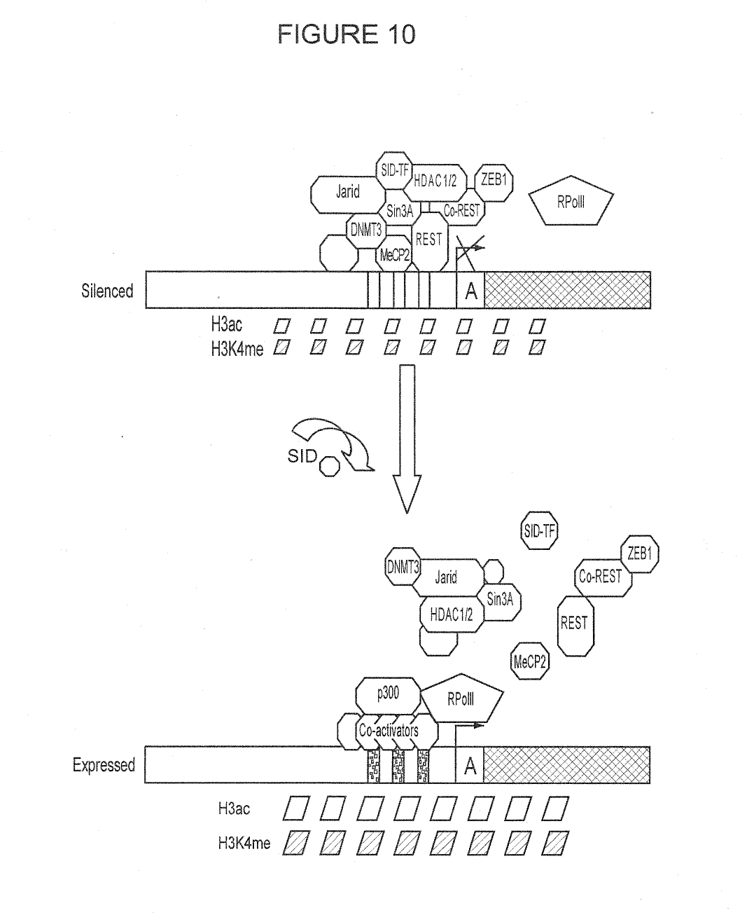

According to certain aspects, use of the compositions and pharmaceutical formulations of the present invention has one or more of the following effects on cancer cells and/or tumors comprising said cancer cells: 1) induces chromatin remodeling and re-expression of silenced genes such as E-cadherin, Estrogen Receptor β, and RARβ, so that the cancer cells more closely resemble normal cells (see FIGS. 5E, 8, and 9); 2) induces breast cancer morphogenesis, differentiation, adherence, contact inhibition; 3) decreases invasive colonies, changing colonies from invasive to non-invasive, thereby preventing metastasis (see FIG. 7); 4) inhibits tumor growth in vitro and in vivo; 5) increases sensitivity to estrogen and retinoids in triple-negative breast cancer; 6) activates histone marker H3K4 ME2; 7) decreases DNA methylation and restores gene function (see FIG. 6); 8) compromises the function of JARID (see FIG. 10); and 9) blocks the function of REST, which compromises the function of ZEB1 (important for its role in maintaining cancer stem cells, a root cause of cancer), allowing re-expression of E-cadherin (see FIG. 10). These factors may affect the aggressiveness of the tumors, their invasiveness, their susceptibility to treatment using one or more therapeutic compositions, etc.

According to further aspects, the compositions and formulations of the present invention may be used so as to render cancer cells and/or tumors comprising said cancer cells more susceptible to treatment using existing therapies, such as hormonal therapy, chemotherapy, retinoid therapy, and tamoxifen therapy, and may also be used to improve the effectiveness of those therapies.

### 4. Methods of Treatment

The present invention also relates to methods for treating and/or preventing cancer by administering the compositions and dosage forms of the present invention in an amount sufficient to treat and/or prevent cancer. The present invention further relates to methods for diagnosing and/or monitoring the progression of cancer by administering labeled compositions and dosage forms of the present invention in an amount sufficient to permit cancer cells and/or tumors to be detected and quantified, either in vivo or in vitro.

The present invention provides methods of administering compositions capable of interacting with the Sin3A/B protein to disrupt its function, and according to further aspects, the compositions are peptides derived from the Sin3A interaction domain (SID). According to other aspects, the compositions include small molecule inhibitors (SMIs), such as peptides derived from the MAD I motif known as the mSin3A interaction domain (SID), where the SMIs are capable of interfering with interactions between the SID and the paired amphipathic helix domain 2 (PAH2) domain of the transcription repressor Sin3A. The present invention further relates to methods for treating cancers, particularly breast cancer, and more particularly triple-negative breast cancer, by administering the compositions or pharmaceutical formulations containing them.

The terms “treat” or “treatment” as used herein may refer to both therapeutic treatment and prophylactic or preventative measures, wherein the object is to prevent or minimize an undesired physiological change or disorder, such as the development or spread of cancer. For purposes of this aspect of the invention, beneficial or desired clinical results include, but are not limited to, alleviation of symptoms, diminishment of extent of disease, stabilized state of disease, delay or slowing of disease progression, amelioration or palliation of the disease state, and remission (whether partial or total), whether detectable or undetectable. For example, treatment with the SID decoy peptides and/or SMIs of the invention may include reduction of undesirable cell proliferation and invasiveness, and/or induction of apoptosis and cytotoxicity. “Treatment” can also mean prolonging survival as compared to expected survival if not receiving treatment. Those in need of treatment include those who already suffer from a cancer, as well as those prone to develop the cancer, or those in whom the cancer is to be prevented or delayed.

The methods of the present invention relate to the administration of an effective amount of the SID decoy peptide and/or SMIs of the present invention. As used herein, the term “(therapeutically) effective amount” refers to an amount of the composition of the invention or other agent (e.g., a drug) effective to treat or prevent cancer (particularly breast cancer) in a mammal. In the case of cancer, the therapeutically effective amount of the agent may reduce (i.e., slow to some extent and preferably stop) unwanted cell proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the administered compositions prevent the growth of and/or kill existing cancer cells, they may be cytostatic and/or cytotoxic.

According to some aspects of the invention, the methods involve administration of a “growth inhibitory amount” of the compositions of the invention. The term “growth inhibitory amount” refers to an amount that inhibits growth or proliferation of a cancer cell, either in vitro or in vivo. The methods of the present invention may inhibit cell growth, for example, by reducing expression of proteins associated with tumor growth and/or invasiveness. In a preferred aspect of the invention, the growth inhibitory amount inhibits (i.e., slows to some extent and preferably stops) proliferation or growth of the target cell in vivo or in vitro by greater than about 25%, preferably greater than about 50%, more preferably greater than about 75%, and most preferably greater than about 85% (e.g., from about 75% to about 100%).

Dosing is dependent on severity and responsiveness of the cancer to be treated, with course of treatment lasting from several days to several months. Optimal dosing schedules can be calculated from measurements of drug accumulation in the body. Persons of ordinary skill can easily determine optimum dosages, dosing methodologies and repetition rates. Optimum dosages may vary depending on the relative potency of individual peptides, and can generally be calculated based on IC50s or EC50s. For example, given the molecular weight of a peptide and an effective dose such as an IC50, for example, a dose in mg/kg can be calculated. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

In accordance with some aspects of the invention, administration of the SID decoy and/or SMI, or pharmaceutical dosage forms containing them, modulates at least the following characteristics of a cancer (such as breast cancer, and particularly triple-negative breast cancer): 1) induces chromatin remodeling and re-expression of silenced genes such as E-cadherin, Estrogen Receptor, and RARβ, so that the cancer cells more closely resemble normal cells (see FIGS. 5E, 8, and 9); 2) induces breast cancer morphogenesis, differentiation, adherence, contact inhibition; 3) decreases invasive colonies, changing colonies from invasive to non-invasive, thereby preventing metastasis (see FIG. 7); 4) inhibits tumor growth in vitro and in vivo; 5) increases sensitivity to estrogen and retinoids in triple-negative breast cancer; 6) activates histone marker H3K4 ME2; 7) decreases DNA methylation and restores gene function (see FIG. 6); 8) compromises the function of JARID (see FIG. 10); and 9) blocks the function of REST, which compromises the function of ZEB1 (important for its role in maintaining cancer stem cells, a root cause of cancer), allowing re-expression of E-cadherin (see FIG. 10). According to further aspects, the modulation of one or more of these characteristics also renders a cancer more susceptible to treatment using existing therapies, such as hormonal therapy, chemotherapy, retinoid therapy, and tamoxifen therapy, and improves the effectiveness of those therapies.

According to certain aspects of the invention, administration of the SID decoy peptide and/or SMI both in vitro and in vivo, and the expression of the sequence corresponding to the SID decoy peptide, can be used to disrupt the interaction between the PAH-2 domain of Sin3 and MAD (and potentially other SID-containing proteins), thus leading to dramatic phenotypic changes in human and mouse breast cancer cells characterized by an early increase in cell adhesion and subsequent contact inhibition. On a molecular level, in MDA-MB-231 cells these changes are associated with induction of E-cadherin and β-catenin. Moreover, MDA-MB-231 cells expressing SID or treated with SID peptide form mainly non-invasive colonies, undergoing morphogenesis and growth inhibition.

The methods of the invention can be carried out in vivo or in vitro, to treat cancer in humans and non-human mammals. The treatment for cancer may be a result of the anti-proliferative activity of the SID decoy peptide and/or SMIs, such as by blocking the interaction of MAD I SID with the PAH2 domain of mSin3A, or by other mechanisms. In one embodiment, the cancer cells are re-induced to express markers that are present in non-cancerous cells as a result of the binding of the SID decoy peptide and/or SMIs.

The methods of the present invention can be advantageously combined with at least one additional treatment method, including but not limited to, chemotherapy, radiation therapy, or any other therapy known to those of skill in the art for the treatment and management of proliferation disorders such as cancer. According to some aspects of the invention, sirtuin inhibitors (and other “epi-drugs”) are used in conjunction with the SID decoy, and the combination may produce a synergistic effect.

Phenotypic changes are a consequence of the methods of the present invention, which disrupt PAH-2 Sin3 mediated functions. As yet it is unclear whether this is specifically due to interference with Sin3 recruitment by SID-containing transcription factors or if the SID decoy can also prevent the binding of certain co-repressor components to the core Sin3 complex, and the invention is not to be considered limited to a particular mechanism of action. Re-expression of the key breast-cancer associated genes CDH1 and ESR1 in MDA-MB-231 cells is accompanied by extensive epigenetic reprogramming, characterized by decreased levels of promoter hypermethylation, increase histone H3K4 methylation (a gene activation mark), increased H3 acetylation, and decrease H3K27 trimethylation. Breast cancer genes such as CDH1, ESR1 and RARβ are re-expressed and function in triple-negative breast cancer cells as a result of the methods of the present invention, forming the basis for a specific and effective treatment for triple-negative breast cancer in particular. For example, the ectopic expression of E-cadherin in MDA-MB-231 cells suppresses their invasive properties.

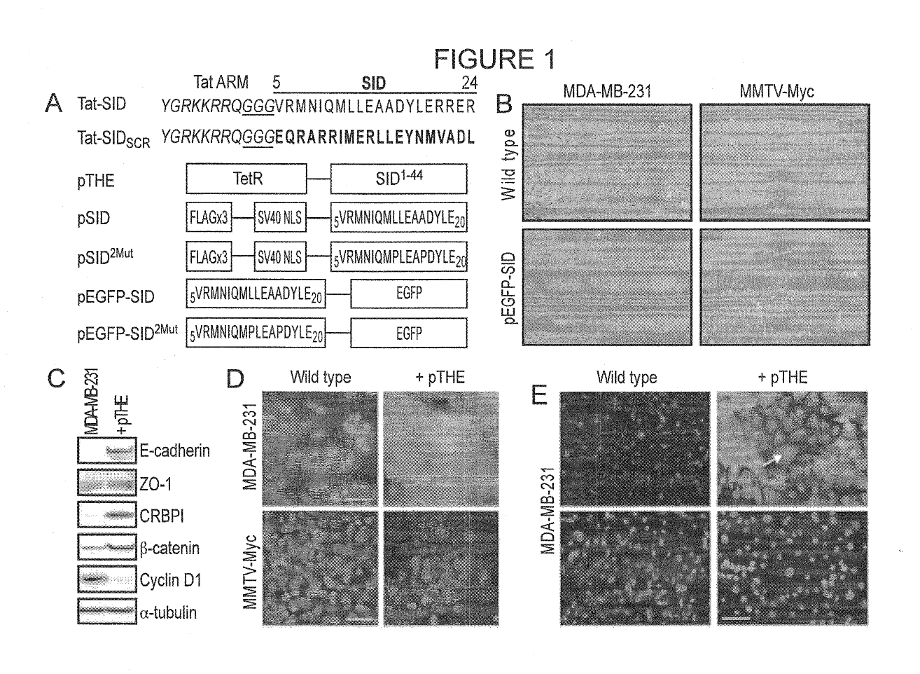

According to one aspect of the invention, a vector that contained the SID (mSin3A interaction domain) of the Mad I motif was constructed, and upon administration was shown to bind with high affinity to the PAH-2 domain of the transcription repressor Sin3A. The transcriptional regulation and function of this defined motif of Sin3A, which is mediated by HDAC1 and HDAC2, was profoundly affected because this interaction is inhibited by SID overexpression. Transfection of the minimal SID or treatment in which the encoded 13 amino acid peptide (SID peptide) bound to the Sin3A PAH-2 domain induced phenotypic changes in human and mouse breast cancer cells in vitro and in vivo. The changes included the induction of differentiated morphogenesis with loss of the invasive colonies on matrigel (see FIG. 7), and increased adhesion, contact inhibition and the expression of differentiation-related proteins such as E-cadherin, p-catinen, p27, CRBP13 and RARα. This was accompanied by anti-proliferative effect in breast cancer cells, but not in immortalized, non-transformed mammary cells in vitro. There was also a marked (70%) inhibition of in vivo tumor growth in mice with minimal toxicity. Mutation of two amino acids in the PAH-2 binding peptide attenuated this activity. Extracts of human breast cancer cells transfected with SID or the addition of a SID-derived peptide in vitro relieved repression of an E-cadherin E-Box reporter and inhibited GST pull-down of in vitro translated Mad I, showing an interference with PAH-2 binding to the specific Mad I transcription factor. Breast cancer cells transfected with SID underwent significant early chromatin remodeling, such as demethylation of the E-cadherin gene. Targeted disruption of the interaction between a specific site of a component of a transcriptional repressor limited to a few transcription factors can be used to induce a differentiation phenotype and anti-tumor effect in breast cancer (see FIG. 1).

The compositions and dosage forms of the present invention may be beneficially used in methods of providing differentiation therapy for a variety of cancers, by targeting specific epigenetic alterations that contribute to the malignant phenotype. The methods of the present invention thereby provide a novel approach to the treatment of cancer, particularly breast cancer, and more particularly triple-negative breast cancer.

These and other aspects of the invention are further described in the non-limiting Examples set forth below.

### Examples

The studies carried out in the Examples described herein were performed to define the molecular basis whereby the SID peptide induced such dramatic changes in human and mouse breast cancer cells in vivo and in vitro. Since the number of transcription factors that are directly affected by SID peptide is relatively small (see FIG. 1), the studies focused on evaluating the gene targets and networks involved, such as over-expression of Myc, which may induce stem cell proliferation, differentiation and senescence.

Materials and Methods

All experiments were conducted with at least three replicates in at least two independent experiments. Statistical analysis: p values were calculated using the unpaired Student's t-test, Mann-Whitney or one-way Anova analysis, as indicated, and were performed using Student's t test.

Plasmids and Constructs

pGEX5X-1-PAH2 was constructed by cloning the DNA sequence corresponding to amino acids 306-450 of mSin3A into the EcoRI and Sall sites of pGEX5X-1 (Amersham). pVP16-MAD was constructed by sub-cloning a HindIII-Notl fragment from fl-VP16-MAD expressing the VP16 activation domain as in-frame fusion with full-length MAD (Ayer et al 1996). pCDNA3.1-MAD was generated by sub-cloning a HindIII-EcoRI fragment from pBJ3-MAD (Amati et al 1993). pGALO-Sin3B, pNLVP16-HDAC1, pT109-GAL4UAS×5-Tk-luc (Guidez et al1998), and pTHE and pTRE (Jiang et al 2001) have been previously described. pSID and pEGFP-SID were constructed using mammalian expression plasmids pCMV-3Tag-1a (Stratagene) and pEGFP-N3 (Clontech), respectively. Two portions of parent vector pEGFP-N3 were extended in parallel reactions with Pfx DNA polymerase (Invitrogen) (one cycle 95° C. 5′, 3-10 cycles 95° C. 30 sec followed by 3 min at 68° C. and 5 min final extension at 68° C.) in the presence of one of two complementary primers:

(SEQ ID No. 6)
5′-GGATCCATCGCCACCATGAACATCCAGATGCTGCTGGAGGCGGCCGA
CTATCTGGAGCCTCCAAAAAAGAAGAGAAAGGTAGTGAGCAAGGGCGAGG
AG-3′;
and
(SEQ ID No. 7)
5′-CTCCTCGCCCTTGCTCACTACCTTTCTCTTCTTTTTTGGAGGCTCCA
GATAGTCGGCCGCCTCCAGCAGCATCTGGATGTTCATGGTGGCGATGGAT
CC-3′

(underlined sequences correspond to SID domain and sequences in bold to SV40 nuclear localization signal, with the remaining sequence corresponding to vector).
Reaction products were then combined and amplified using the same conditions for 25 cycles. Original plasmid DNA was degraded by digestion with Dpnl. pEGFP-SID2Mut was made by subjecting pEGFP-SID to the same mutagenesis protocol; as above using the following primers:

(SEQ ID No. 8)
5′- CACCATGAACATCCAGATGCCGCTGGAGGCGCCCGACTATCTGGAG
CCTCCA-3′;
and
(SEQ ID No. 9)
5′- TGGAGGCTCCAGATAGTCGGGCGCCTCCAGCGGCATCTGGATGTTC
ATGGTG-3′

(mutated nucleotides are indicated in bold).
To make pSID and pSID2Mut, the corresponding sequences from pEGFP-SID and pEGFP-pSID2Mut were amplified with Pfx DNA polymerase (Invitrogen) using primers containing BamHI and Pstl restriction sites and sub-cloned using standard protocols. The primers used were 5′-GCGGGGATCCATGAACATCCAGAT-3′ (SEQ ID No. 10) and 5′-GGGGCCTGCAGTACCTTTCTCTTCTT-3′ (SEQ ID No. 11).

Peptides

An SID peptide (YGRKKRRQGGGVRMNIQMLLEAADYLERRER, SEQ ID No. 12) and control peptide (YGRKKRRQGGGEQRARRIMERLLEYNMVADL, SEQ ID No. 13) were synthesized to a purity level of 94% and 84%, respectively, as assessed by analytical reversed phase-high performance liquid chromatography (Mimotopes).

Cell Lines

293T and triple-negative MDA-MB-231 mammary tumor cells were obtained from the American Tissue Culture Collection (ATCC). 293T and MDA-MB-231 cells were maintained in Dulbecco medium supplemented with 10% fetal calf serum. MMTV-Myc cell lines were generated from fragments of tumors from untreated MMTV-c-Myc mice after digestion with collagenase (1.5 mg/ml collagenase, 25 mg/ml BSA in PBS with Ca2+ and Mg2+) at 37° C. for 30-45 min with gentle agitation. MMTV-Myc were propagated in DMEM/F12 medium supplemented with 10% fetal bovine serum, 5 mg/ml insulin, 1% pen/strep, 1% glutamine and 1% Hepes.

MDA-MB-231 cells stably expressing pTRE, pTHE, pSID, pSID2Mut, pEGFP-SID or pEGFP-pSID2Mut were obtained by transfection with the AMAXA nucleofector system (Amaxa Biosystems) followed by antibiotic selection 48 hrs post-transfection with 300 μg/ml G418 (Roche).

Immunoblotting

Cell lysates were obtained using ice-cold RIPA buffer (Sigma) in presence of proteinase inhibitor cocktail (Complete, Roche), 10 mg/ml PMSF and the phosphatase inhibitors sodium orthovanadate (100 mM) and sodium fluoride (100 mM). Protein samples were analyzed according to standard sodium dodecyl sulfate-polyacrylamide gel electrophoresis (SDS-PAGE) and Western immunoblotting protocols. The antibodies used were as follows: anti E-cadherin and ZO-1 antibodies (Zymed), anti β-catenin (Cell Signaling, Technologies), anti-Cyclin D1 (Santa Cruz Biotechnology) and anti-Tubulin (Sigma).

GST Pull-Down Assay

GST pull-down assays were performed using the ProFound™ pull-down GST protein:protein interaction kit (Pierce) according to the manufacturer's instructions. GST and GST-Sin3A PAH2 were prepared in E. coli using standard procedures (Dong et al 1996). 35S-methionine-labeled MAD was synthesized in vitro using a rabbit reticulocyte coupled transcription-translation system (Promega), following the supplier's directions. MAD immunoprecipitates were prepared from MDA-MB-231 cell lysates using rabbit polyclonal anti-MAD (C-19) antibody (Santa Cruz) the Catch and Release v2.0 Reversible Immunoprecipitation System (Millipore), following the manufacturer's instructions. Samples containing 35S-labeled in vitro translated or immunoprecipitated MAD were incubated with 100 μg GST or a given GST fusion protein. Where samples were treated with peptide (1.5 μM or 15 μM), bait protein (GST or GST-PAH) and prey protein (in vitro translated or immunoprecipitated MAD) were pre-incubated separately for 1 hour at 4° C. prior to the pull-down assay being performed. Bound proteins were eluted in glutathione elution buffer, diluted in Laemmeli loading buffer and separated by SDS-PAGE using Next Gel (Amresco). Gels containing 35S-labeled samples were fixed in 25% isopropanol and 10% acetic acid, dried, and exposed to Biomax MR film (Kodak). Gels containing samples from MDA-MB-231 cell lysates were analyzed by Western immunoblot according to standard protocols. Pulled-down MAD was detected using mouse monoclonal anti-MAD (F-1) antibody (Santa Cruz), anti-mouse secondary antibody (Jackson ImmunoResearch), and enhanced chemiluminescence (ECL, Peirce).

Immunofluorescence

MDA-MB-231 and MMTV-Myc cells were cultured on 8 chambered wells (Fisher, Pa.) and fixed with 3% paraformaldehyde/PBS for 15 min. at RT. Autofluorescence was quenched by incubation in 50 mM NH4Cl for 5 min. Cells were permeabilized with 0.2% Triton X-100/PBS and blocked with 2% BSA and 1:100 goat anti-mouse F (ab′)2 fragment (Jackson ImmunoResearch, PA) in PBS for 40 minutes. Primary antibodies, E-cadherin and b-catenin (Zymed) were incubated overnight at 4° C. in blocking buffer and washed 3 times with washing buffer (0.05% Triton X-100/PBS) and once with PBS. Anti mouse or rabbit-Alexa 488 or 568 were used as secondary antibodies in a dilution 1:200 in blocking buffer and washed. The samples were then incubated with DAPI (Sigma) 10 min. Finally, samples were washed twice with PBS and once with water and mounted with ProLong Gold antifade reagent (Molecular Probes/Invitrogen, CA) following the manufacturer instructions. All incubations and washes were done at 4 or 25° C. as required, with gentle agitation in an Enviro-Genie rocking incubator (Scientific Industries, NY) to reduce the variability between assays. Confocal microscopy was performed using a Leica TCS-SP (UV) confocal microscope at the Mount Sinai School of Medicine's Microscopy Shared Resource Facility.

Three-Dimensional Morphogenesis Assay

3D cultures of MDA-MB-231 and MMTV-Myc cells stably transfected with pEGFP-SID, pEGFP-SID2Mut or the parent vector pEGFP-N3 were prepared as described by Debnath et al. (Debnath et al2003). Briefly, 75 ml/well of growth factor depleted Matrigel (BD Biosciences) was applied on 8-well multichamber slides (BD Biosciences) and incubated at 37° C. for 30 minutes. Once the Matrigel solidified 5×103 cells were seeded on top of the gels in assay medium containing 2% Matrigel and 20 ng/ml epithelial growth factor. Growth medium was changed daily for 14 days. Once colony formation was evident, the SID or the scramble control peptide was added to the MDA-MB-231 parental cells in the medium starting at day 3. The morphology of the colonies was determined by confocal microscopy. Induction of apoptosis was determined by staining colonies with activated caspase-3 antibody (BD Transduction Labs) and cell polarization by staining with anti-GM130 antibody (BD Transduction Labs). Cell nuclei were visualized by DAPI (Sigma) staining.

Proliferation Assay

After cell attachment wild type MDA-MB-231 cells or MDA-MB-231 cells stably transfected with pTHE plasmid were stimulated overnight with 2.5 nM estrogen (E2) or E2 plus 2.5 mM tamoxifen to determine ERα activation. For activation of RARs 1 mM ATRA (all-trans-retinoic acid, pan-RAR agonist) or 100 nM Am580 (retinoibeozoic acid, RARα-specific agonist) was used daily for 48 hours. Cell proliferation was determined by MTT (Thiazolyl Blue Tetrazolium Bromide) colorimetric assay following standard protocols (Invitrogen). The optical density (OD) of reduced MTT was assessed at 570 nm versus 620/630 nm after 4 hr at 37° C. Assays were performed in quadruplicate.

Mammalian Two-Hybrid Assay

24 hours prior to transfection, 293T cells were plated at 50-60% confluence on 48-well plates. Cells were transfected with 0.2 μg of plasmid DNA and 2 μl of Polyfect (Qiagen) following the manufacturer's recommended protocol. 48 hours post-transfection luciferase activity was measured using the Dual Luciferase assay system (Promega) on a Mithras LB940 plate reader (Berthold Technologies). The pRL-TK renilla luciferase vector (5 ng per well) was co-transfected to correct for variations in transfection efficiency.

Quantitative Real-Time PCR

Total RNA stabilized with RNAlater (Ambion) was isolated using RNeasy Mini Kit (Qiagen) and cDNA was prepared using the High Capacity cDNA Reverse Transcription Kit (Applied Biosytems), all following manufacturers' instructions. Quantitative real-time PCR was performed using Mesa Fast qPCR MasterMix Plus for SYBR Green (Eurogentec) on 7900HT Fast real Time PCR system (Applied Biosytems) using the following conditions: first a denaturation step at 95° C. for 5 minutes followed by 40 cycles of denaturation at 95° C. for 5 seconds, annealing at 60° C. for 30 seconds and elongation at 72° C. for 30 seconds. For determination of gene expression, values were shown in molecules per μg total RNA and were derived from the ΔCt between the GAPDH housekeeping gene and the gene of interest. The amount of GAPDH molecules per pg total RNA was determined by absolute quantification. The PCR primers used were:

| (SEQ ID No. 14) | |
|---|---|
| RT-CDH1-Fwd | 5′-CCGCTGGCGTCTGTAGGA-3′; |
| (SEQ ID No. 15) | |
| RT-CDH1-Rev | 5′-AGGGCTCTTTGACCACCGCTCT-3′; |
| (SEQ ID No. 16) | |
| RT-ESR1-Fwd | 5′-GATCCACCTGATGGCCAAG-3′; |
| (SEQ ID No. 17) | |
| RT-ESR1-RT | 5′-ACAGATGCTCCATGCCTTTG-3′; |
| (SEQ ID No. 18) | |
| RT-RPL30-Fwd | 5′-GACAAGGCAAAGCGAAATTG-3′; |
| (SEQ ID No. 19) | |
| RT-RPL-Rev | 5′-GTATTTTCCGCATGCTGTGC-3′, |
| (SEQ ID No. 20) | |
| RT-GAPDH-Fwd | 5′-ATGGGGAAGGTGAAGGTCG-3′; |
| and | |
| (SEQ ID No. 21) | |
| RT-GAPDH-Rev | 5′-TAAAAGCAGCCCTGGTGACC-3′. |

Chromatin Immunoprecipitation (ChIP)

ChIP was performed on MDA-MB-231 cells using the EpiQuik™ chromatin immunoprecipitation kit (Epigenetik), according to standard protocols and the supplier's directions. Cross-linked cells were sonicated using a Bioruptor (Diagenode) and samples containing 1×106 cells were immunoprecipitated with antibodies directed against dimethyl H3 Lys4 (Active Motif), trimethyl H3 Lys4 (ab8580, Abcam), trimethyl H3 Lys27 (Millipore) or acetyl-histone H3 (Millipore). DNA obtained from input or immunoprecipitated DNA was analyzed by real-time PCR using primers mapping to the CDH1 and ESR1 promoter regions. The promoter of the RPL30 housekeeping gene was used a control. Quantitative real-time PCR was performed as described above. To quantify chromatin immunoprecipitates, a standard curve was generated from serial dilutions of 10% input DNA. The following PCR primers were used:

(SEQ ID No. 22)

| | |
|---|---|
| ChIP-CDH1-Fwd | 5′-CCCTCTCAGTGGCGTCGGAACT-3′; |

(SEQ ID No. 23)

| | |
|---|---|
| ChIP-CDH1-Rev | 5′-GTACCTGCAGCAGCAGCAGCA-3′; |

(SEQ ID No. 24)

| | |
|---|---|
| ChIP-ESR1-Fwd | 5′-ATGAGCTCGGGAGACCAGTA-3′; |

(SEQ ID No. 25)

| | |
|---|---|
| ChIP-ESR1-Rev | 5′-GCTGTGCTCTTTTTCCAGGT-3′, |

(SEQ ID No. 26)

| | |
|---|---|
| ChIP-RPL30-Fwd | 5′-GCAAAGCGAAATTGGTCATT-3′; |
| and | |

(SEQ ID No. 27)

| | |
|---|---|
| ChIP-RPL-Rev | 5′-CTGTTTTCACTCCTGCCACA-3′. |

Bisulphite Assay

Bisulfite modification was performed using the MethylEasy Xceed kit (Human Genetic Signatures) following to the manufacturer's protocol and PCR primers were selected using MethPrimer software. The following PCR primers were used:

| (SEQ ID No. 28) | |
|---|---|
| BS-CDH1-Fwd | 5′-AATAAAAGAATTTAGTTAAGTGT-3′; |
| (SEQ ID No. 29) | |
| BS-CDH1-Rev | 5′-AATACCTACAACAACAACAAC-3′; |
| (SEQ ID No. 30) | |
| BS-ESR1-Fwd | 5′-TGTTTGGAGTGATGTTTAAGTT-3′; |
| and | |
| (SEQ ID No. 31) | |
| BS-ESR1-RT | 5′-ATAAAACCATCCCAAATACTTT-3′. |

PCR was performed using Amplitaq Gold 360 DNA Polymerase (Applied Biosystems) supplemented with GC Enhancer. PCR products were separated by agarose gel electrophoresis, cloned into the pCR4-TOPO vector (Invitrogen) and sequenced on a 3730 DNA Analyzer (Applied Biosystems) using the T7 forward primer.

Statistical Analyses

Statistical analyses were performed with GraphPad Prism software (version 5.0). Where shown, p values were calculated using the unpaired Student's t-test, Mann-Whitney or one-way Anova analysis as indicated.

SID Over Expression Induces Growth Inhibition and Differentiation of Breast Cancer Cells.

To interfere with the Sin3 PAH2 domain interaction with their partners we constructed 2 vectors and the pTHE vector containing a specific motif called SID (mSin3A interaction domain) of MAD, which binds with high affinity to the PAH2 domain of the transcription repressor Sin3 (FIG. 1A). We also designed cell-penetrating peptides encoding the SID amino acid sequence and a scrambled control sequence, to target the PAH2 domain (FIG. 1A). Dramatic phenotypic changes were observed in human MDA-MB-231 and MMTV-Myc mouse breast cancer cells carried in culture following stable transfection with the 3 vectors expressing SID or treated with the SID peptide switching from a spindle shape morphology with no defined cell-cell contacts to cobblestone monolayers and well defined cell-cell contact accompanied by cell growth contact inhibition (FIG. 1B). Biochemical analysis showed an increase in the expression of E-cadherin, membrane-associated β-catenin and ZO-1, all of which are involved in cell contact inhibition (FIG. 1C). Other differentiation related proteins were induced such as nuclear localized RARβ (FIG. 1E) and CRBP1 (FIG. 1C) both RARα target genes. Consistent with growth arrest was a marked reduction of cyclin D1, (FIG. 1C). No changes in cell morphology, growth or survival were observed in the E-cadherin positive MCF-10A immortalized normal breast cell line transfected with any of the construct expressing SID, suggesting that these changes in the phenotype are specific to transformed cells (FIG. 6).

SID and SID Peptide Block the Interaction Between MAD SID Domain and PAH-2 domain of Sin3 and Inhibit Sin3 Activity.

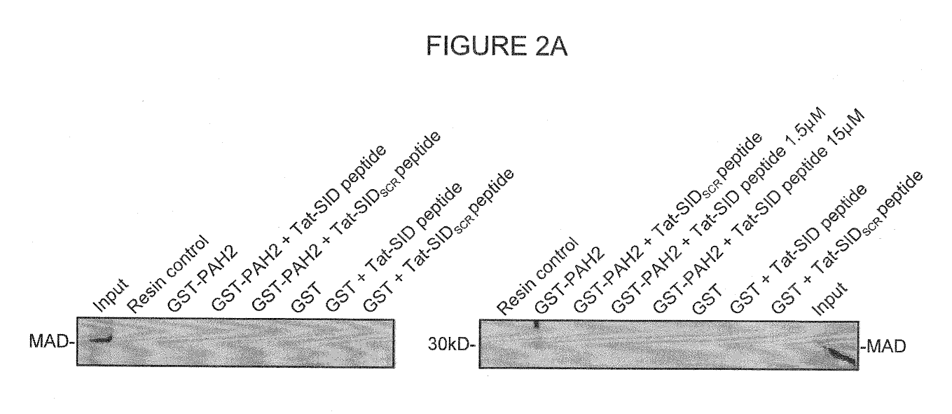

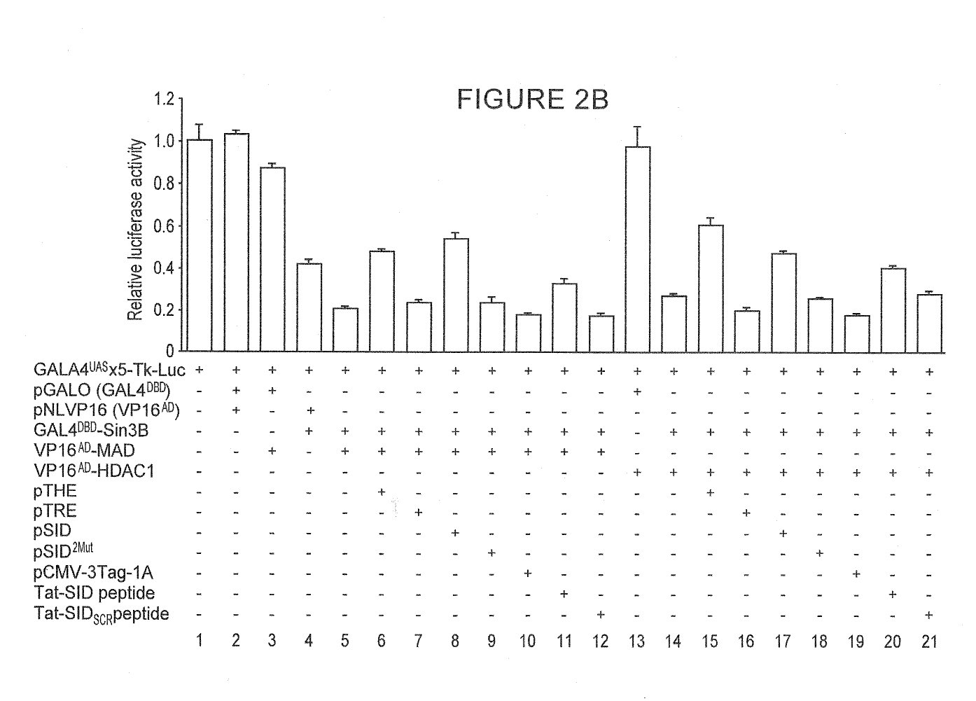

To confirm that the presence of the MAD SID decoy peptide or expression of the SID sequence (FIG. 1A) blocked the interactions between Sin3 PAH2 and partner proteins we tested its activity in both in vitro and in vivo models (FIG. 2A). As predicted, the introduction of SID peptide but not a scrambled control blocked interactions between the PAH2 domain of Sin3A and both in vitro translated MAD (FIG. 2A) and MAD immunoprecipitated from SID peptide treated MDA-MB-231 cells (FIG. 2A, right panel). This result was confirmed in 293Tcells using the mammalian two-hybrid system (FIG. 2B). Co-expression of GAL4 DBD-Sin3B and VP16AD-MAD caused a 5-fold decrease in luciferase activity on a reporter under the control of GAL4UAS elements (FIG. 2B, column 5). This repression, despite the presence of the VP16 activation domain fused to MAD, is consistent with previously reported data. Consistent with the in vitro data, expression of the MAD SID peptide sequence from vectors encoding FLAG-tagged SID and a fusion of TetR and SID led to a 2-fold increase in luciferase activity compared to that found with GAL4 DBD-Sin3B and VP16AD-MAD co-expression (FIG. 2B, columns 6 and 8). This result was also confirmed, albeit to a lesser degree by applying SID peptide to the cell culture medium (FIG. 2B, column 11). Neither co-expression of doubly mutated SID, nor addition of scrambled control peptide led to an increase in luciferase activity. In order to determine whether the SID decoy peptide interfered exclusively with interactions between Sin3 PAH2 and its partner proteins, or had a wider effect on assembly of the Sin3 co-repressor complex we also tested the effect of the SID decoy on luciferase activity resulting from co-expression of GAL4 DBD-Sin3B and VP16AD-HDAC1 (FIG. 2B, columns 14-21). Interestingly, the data obtained were very similar to those for GAL4 DBD-Sin3B and VP16AD-MAD. This was found for the TetR-SID fusion (28.4 kDa) (FIG. 2B, column 15) but also to a somewhat lesser degree the smaller FLAG-tagged SID (6.6 kDa) (FIG. 2B, column 17), and also the SID peptide (FIG. 2B, column 20). Thus the SID decoy peptide can effect not only binding of Sin3-PAH2 partner proteins but also recruitment of HDAC1 and, potentially, additional factors that do not interact with Sin3 via the PAH2 domain.

Induction of Morphogenesis by the SID Peptide in Triple Negative Breast Cancer Cells.

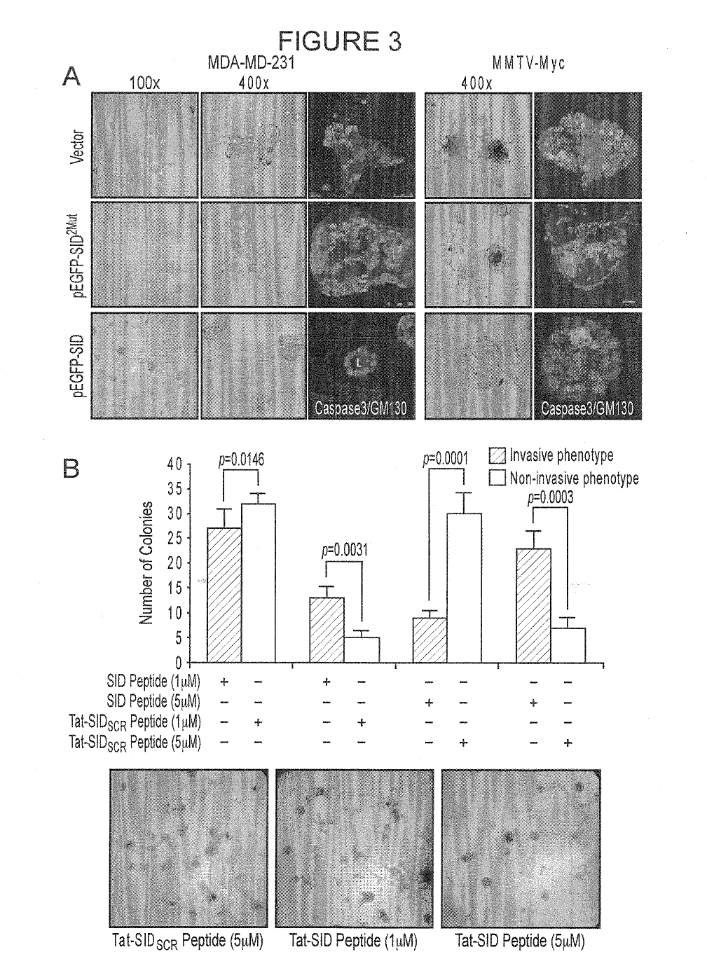

In order to determine the extent of the reversion of the transformed phenotype in MDA-MB-231 and MMTV-Myc cells 3D-cultures in basement membrane matrix (Matrigel, BD Biosciences) were prepared to examine the morphogenetic potential of the SID peptide expression in these cell lines. Stable transfected cells expressing the empty vector, the SID amino acid sequence (SID) or a double mutant incapable to bind the PAH2 domain of Sin3 (SID-L12P/A16P) were seeded in quadruplicates (5×103 cells/well) on Matrigel following the protocol described by Debnath et al. Phase contrast and confocal IF indicated that the highly invasive phenotype exhibited by both cell lines (star like colonies) was blocked only in SID expressing cells (smooth, round colonies). The quantification of this (5 fields/well) indicates that about only 15% of the colonies retained a mild invasive phenotype (FIG. 3A). The confocal analysis revealed that about 25% of the MDA-MB-231 SID expressing colonies underwent acinar morphogenesis but with poor polarization (FIG. 3A middle panel). Although the MMTV-Myc cells displayed a strong reduction in the invasive phenotype few colonies showed rudimentary lumen formation with abnormal polarization (FIG. 3A, right panel). Similar results were obtained by treating the MDA-MB-231 cells with the cell penetrating SID peptides, interestingly, besides decreasing the number of the colonies with invasive phenotype by ˜80%, the number and size of the colonies were reduced when cultures were exposed to 5 μM SID (FIG. 3B, lower panel). As expected the negative control scramble (SCR) peptide did not exert this effect. These results indicate that it is possible to induce a substantial degree of differentiation and morphogenesis in triple negative cell lines by selective interference with the Sin3 activity.

SID Induces Anti-Tumor Effect In Vivo.

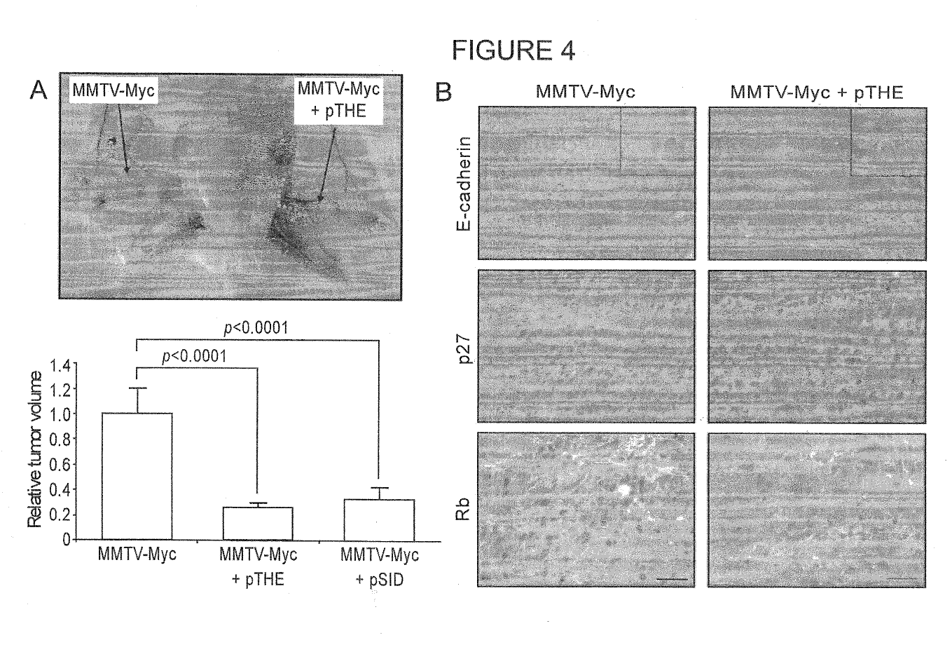

To study the in vivo anti-proliferative effect of SID decoy, MMTV-Myc cells with stable SID expression were generated from MMTV-c-Myc tumors. MMTV-Myc cells stably expressing pTHE plasmid or pEGFP-SID were injected into the fat pads of FVB syngeneic mice. There was a 75% decrease in relative tumor volume 14 days later as compared to the vector control (FIG. 4A, p<0.0001). Immunohistochemical analysis of tumors revealed that the introduction of SID decoy induced re-expression of membrane-associated E-cadherin, increased expression of the cyclin-dependent kinase inhibitor p27Kip1 (a marker of growth arrest) and down-regulated pRB in all animals examined (n=8) (FIG. 4B). Caspase-3 staining revealed no differences in apoptosis, suggesting that the anti-tumor effect of the SID interference is mainly associated with diminished invasiveness and induction of terminal differentiation rather than via apoptosis.

SID Domain Expression Induces Early and Intense Chromatin Remodeling.

Given that expression of the SID decoy peptide is associated with re-expression of genes silenced in MDA-MB-231 cells, we sought to identify epigenetic changes associated with the promoter regions of two genes that are important in the pathology of breast cancer, CDH1 and ESR1, which encode e-cadherin and ERα, respectively. We found that while there was a modest increase in the overall acetylation levels of histone H3, the levels of H3K4 methylation increased dramatically, in particular for CDH1 (FIG. 5B). This was accompanied by a large increase in the levels of E-cadherin and ERα expression (FIG. 5C). Similar results on H3K4 methylation were observed for RARβ, consistent with the re-expression shown in FIG. 1E (data not shown). These changes were not found in the RPL30 housekeeping gene used as control. Consistent with the notion that epigenetic reprogramming of silenced gene promoters occurs in response to SID decoy, compared to wild-type cells bisulfite sequencing of the ESR1 promoter/5′UTR revealed an average 70% loss of CpG methylation (p=0.0095) within this region in MDA-MB-231 cells stably transfected with pSID but not pSID2Mut (FIG. 5D, right panel). Bisulfite sequencing analysis of the CDH1 promoter/5′UTR showed that compared to pSID2Mut-transfected or wild-type MDA-MB-231 cells there was also an average 60% decrease in the level of CpG methylation (p=0.0031) in pSID-transfected cells. Interestingly, in the cases of both the ESR1 and CDH1 promoter/5′UTRs the most significant loss of CpG dinucleotide demethylation was focused on regions adjacent to the transcription initiation sites (FIG. 5D, left panel).

SID Induces of ER and RARβ Sensitivity to Estrogen and Retinoids.

The transfection of MDA-MB-231 cells with two different SID constructs or SID peptide induced the re-expression of ERα and RARβ (FIG. 5B). Functionality of the ER re-expression was demonstrated by induced growth sensitivity to 2.5 μM estradiol (E2) treatment of MDA-MB-231 cells and the prevention of E2 growth stimulation by 2.5 μM tamoxifen as measured by MTT assay (FIG. 5E, left panel). The progesterone receptor a target gene of ERα, was also induced by SID (not shown). Immunofluorescence analysis also shows an increased expression of RARβ in cells transfected with SID (FIG. 1E). This finding coincides with the increased expression of E-cadherin and CRBP1, known RAR target genes and the induction of p27, highly sensitive to RAR activation. Furthermore, the re-expression of RARβ is functional since there is a significant growth inhibition of MDA-MB-231 cells by treatment with the retinoids ATRA (1 uM) and RARα-specific agonist AM580 (100 nM) (FIG. 5E, right panel), similar results were obtained using T-47D and MCF-7 human breast cancer cells, which basally express RARβ (data not shown). Thus, blocking specific sites of a component of a transcriptional repressor limited to few transcription factors can be used to induce a differentiation phenotype and anti-tumor effect in breast cancer and that these events may, impart new and/or restore therapeutic targets in breast cancer.

While the increase in histone H3 acetylation found with expression of the SID decoy is in line with the well-established histone deacetylase activity associated with Sin3, such large increases in levels of H3K4 methylation were not expected. However, recent research has demonstrated a role for Sin3 and JARID1A (RBP2/KDM5A), a H3K4me2/me3 demethylase, in permanent gene silencing in myoblasts. It was shown that Sin3 and JARID1A directly interact and that JARID1A is associated with the CDH1 promoter. Another JARID family member, JARID1B (PLU-1/KDM5B), which shares a high degree of homology with JARID1A is also of great interest. While a direct interaction with Sin3 remains to be tested, JARID1B does interact directly with histone deacetylases and it has been found to be overexpressed in breast cancers. Furthermore, the Drosophila homolog of JARID1A/1B, LID (KDM5) interacts directly with Sin3 in gene-selective silencing. Both JARID1A and 1B are expressed in MDA-MB-231 cells. However, it is perhaps most noteworthy that in a syngeneic mouse mammary tumor model similar to the one used here, stable knockdown of JARID1 B also resulted in reduced tumor cell growth. Thus blocking the activity of JARID1A/B represents a strong potential candidate mechanism to explain the dramatic increases in H3K4 methylation found with SID decoy expression. While single treatment with inhibitors of class I and II HDAC had very limited effects in MDA-231 cells, it has been shown that use of inhibitors of class III HDACs (Sirtuins) leads to re-expression of E-cadherin in MDA-MB-231 cells. The re-expression of another silenced gene, SFRP1, in MDA-MB-231 cells was not accompanied by a loss of promoter CpG hypermethylation. Because the promoters of both CDH1 and ESR1 are not densely methylated, and loss of DNA methylation following exposure to SID decoy is most apparent in the regions adjacent to the transcription initiation sites. Therefore, the loss of DNA methylation may follow changes in H3K4 methylation status and, as described for Sirtuin inhibitors, re-expression of ESR1 and CDH1 in this case may not be strongly dependent on the removal of methylated CpGs.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

What is claimed:

1. An SID decoy peptide derived from the mSin3A interaction domain (SID), wherein said SID decoy peptide blocks interactions between the PAH2 domain of Sin3A and SID-containing proteins, and wherein said SID decoy peptide is from 13 to 20 amino acids long.

2. The SID decoy peptide of claim 1, wherein the isolated SID peptide comprises VRMNIQMLLEAADYLERRER (SEQ ID No. 1).

3. The SID decoy peptide of claim 1, wherein the isolated SID peptide comprises MNIQMLLEAADYLE (SEQ ID No. 2).

4. The SID decoy peptide of claim 1, wherein the isolated SID peptide comprises MNIQMLLEAPDYLE (SEQ ID No. 3).

5. The SID decoy peptide of claim 1, wherein the isolated SID peptide comprises MNIQMPLEAPDYLE (SEQ ID No. 4).

6. The SID decoy peptide of claims 1-5, wherein the isolated SID peptide further comprises a leader sequence comprising YGRKKRRQGGG (SEQ ID No. 5).

7. A pharmaceutical dosage form comprising the SID decoy peptide of claims 1-6 and one or more pharmaceutically acceptable excipients.

8. The pharmaceutical dosage form of claim 7, wherein the dosage form is adapted for administration via a route selected from the group consisting of enteral, parenteral, intravenous, intramuscular, intrathecal, topical, and subcutaneous administration.

9. Use of the SID decoy peptide of claims 1-6 to modulate factors relating to tumor aggressivity and susceptibility to chemotherapy, the factors selected from the group consisting of:
inducing chromatin remodeling and re-expression of silenced genes selected from the group consisting of E-cadherin, Estrogen Receptor β, and RARβ;
inducing breast cancer cell morphogenesis, differentiation, adherence, and contact inhibition;
decreasing invasive colonies, and changing colonies from invasive to non-invasive, thereby preventing metastasis;
inhibiting tumor growth in vitro and in vivo;
increasing sensitivity to estrogen and retinoids in triple-negative breast cancer;
activating histone marker H3K4 ME2;
decreasing DNA methylation and restoring gene function;
compromising the function of JARID; and
blocking the function of REST, thereby compromising the function of ZEB1 and allowing re-expression of E-cadherin.

10. A method of treating breast cancer, comprising administering an effective amount of at least one SID decoy peptide derived from the mSin3A interaction domain (SID), wherein said SID decoy peptide blocks interactions between the PAH2 domain of Sin3A and SID-containing proteins, and wherein said SID decoy peptide is from 13 to 20 amino acids long.

11. The method of claim 10, wherein the breast cancer is triple-negative breast cancer.

12. The method of claim 10, wherein the growth of breast cancer cells is inhibited.

13. The method of claim 10, wherein binding of SID-partner proteins to PAH2 is inhibited.

14. The method of claim 10, wherein the breast cancer cells are induced to express a receptor selected from the group consisting of E-cadherin, estrogen receptor alpha, retinoic acid receptor beta, and combinations thereof.

15. The method of claim 10, wherein tumor growth is inhibited.

16. The method of claim 10, wherein sensitivity to therapeutic agents selected from the group consisting of 17β-estradiol, tamoxifen, retinoids, and combinations thereof is restored.

17. The method of claim 10, wherein tumor invasiveness is reduced.


SEQUENCE LISTING (33 sequences)

SEQ ID NO: 1 - protein, 20 aa, Artificial Sequence
  Description of Artificial Sequence Synthetic peptide
VRMNIQMLLEAADYLERRER

SEQ ID NO: 2 - protein, 14 aa, Artificial Sequence
  Description of Artificial Sequence Synthetic peptide
MNIQMLLEAADYLE

SEQ ID NO: 3 - protein, 14 aa, Artificial Sequence
  Description of Artificial Sequence Synthetic peptide
MNIQMLLEAPDYLE

SEQ ID NO: 4 - protein, 14 aa, Artificial Sequence
  Description of Artificial Sequence Synthetic peptide
MNIQMPLEAPDYLE

SEQ ID NO: 5 - protein, 11 aa, Artificial Sequence
  Description of Artificial Sequence Synthetic peptide
YGRKKRRQGGG

SEQ ID NO: 6 - DNA, 99 bp, Artificial Sequence
  Description of Artificial Sequence Synthetic primer
ggatccatcgccaccatgaacatccagatgctgctggaggcggccgactatctggagcctccaaaaaagaagagaaaggtagtgagcaagggcgaggag

SEQ ID NO: 7 - DNA, 99 bp, Artificial Sequence
  Description of Artificial Sequence Synthetic primer
ctcctcgcccttgctcactacctttctcttcttttttggaggctccagatagtcggccgcctccagcagcatctggatgttcatggtggcgatggatcc

SEQ ID NO: 8 - DNA, 52 bp, Artificial Sequence
  Description of Artificial Sequence Synthetic primer
caccatgaacatccagatgccgctggaggcgcccgactatctggagcctcca

SEQ ID NO: 9 - DNA, 52 bp, Artificial Sequence
  Description of Artificial Sequence Synthetic primer
tggaggctccagatagtcgggcgcctccagcggcatctggatgttcatggtg

SEQ ID NO: 10 - DNA, 24 bp, Artificial Sequence
  Description of Artificial Sequence Synthetic primer
gcggggatccatgaacatccagat

SEQ ID NO: 11 - DNA, 26 bp, Artificial Sequence
  Description of Artificial Sequence Synthetic primer
ggggcctgcagtacctttctcttctt

SEQ ID NO: 12 - protein, 31 aa, Artificial Sequence
  Description of Artificial Sequence Synthetic polypeptide
YGRKKRRQGGGVRMNIQMLLEAADYLERRER

SEQ ID NO: 13 - protein, 31 aa, Artificial Sequence
  Description of Artificial Sequence Synthetic polypeptide
YGRKKRRQGGGEQRARRIMERLLEYNMVADL

SEQ ID NO: 14 - DNA, 18 bp, Artificial Sequence
  Description of Artificial Sequence Synthetic primer
ccgctggcgtctgtagga

SEQ ID NO: 15 - DNA, 22 bp, Artificial Sequence
  Description of Artificial Sequence Synthetic primer
agggctctttgaccaccgctct

SEQ ID NO: 16 - DNA, 19 bp, Artificial Sequence
  Description of Artificial Sequence Synthetic primer
gatccacctgatggccaag

SEQ ID NO: 17 - DNA, 20 bp, Artificial Sequence
  Description of Artificial Sequence Synthetic primer
acagatgctccatgcctttg

SEQ ID NO: 18 - DNA, 20 bp, Artificial Sequence
  Description of Artificial Sequence Synthetic primer
gacaaggcaaagcgaaattg

SEQ ID NO: 19 - DNA, 20 bp, Artificial Sequence
  Description of Artificial Sequence Synthetic primer
gtattttccgcatgctgtgc

SEQ ID NO: 20 - DNA, 19 bp, Artificial Sequence
  Description of Artificial Sequence Synthetic primer
atggggaaggtgaaggtcg

SEQ ID NO: 21 - DNA, 20 bp, Artificial Sequence
  Description of Artificial Sequence Synthetic primer
taaaagcagccctggtgacc

SEQ ID NO: 22 - DNA, 22 bp, Artificial Sequence
  Description of Artificial Sequence Synthetic primer
ccctctcagtggcgtcggaact

SEQ ID NO: 23 - DNA, 21 bp, Artificial Sequence
  Description of Artificial Sequence Synthetic primer
gtacctgcagcagcagcagca

SEQ ID NO: 24 - DNA, 20 bp, Artificial Sequence
  Description of Artificial Sequence Synthetic primer
atgagctcgggagaccagta

SEQ ID NO: 25 - DNA, 20 bp, Artificial Sequence
  Description of Artificial Sequence Synthetic primer
gctgtgctctttttccaggt

SEQ ID NO: 26 - DNA, 20 bp, Artificial Sequence
  Description of Artificial Sequence Synthetic primer
gcaaagcgaaattggtcatt

SEQ ID NO: 27 - DNA, 20 bp, Artificial Sequence
  Description of Artificial Sequence Synthetic primer
ctgttttcactcctgccaca

SEQ ID NO: 28 - DNA, 23 bp, Artificial Sequence
  Description of Artificial Sequence Synthetic primer
aataaaagaatttagttaagtgt

SEQ ID NO: 29 - DNA, 21 bp, Artificial Sequence
  Description of Artificial Sequence Synthetic primer
aatacctacaacaacaacaac

SEQ ID NO: 30 - DNA, 22 bp, Artificial Sequence
  Description of Artificial Sequence Synthetic primer
tgtttggagtgatgtttaagtt

SEQ ID NO: 31 - DNA, 22 bp, Artificial Sequence
  Description of Artificial Sequence Synthetic primer
ataaaaccatcccaaatacttt

SEQ ID NO: 32 - protein, 16 aa, Artificial Sequence
  Description of Artificial Sequence Synthetic peptide
VRMNIQMLLEAADYLE

SEQ ID NO: 33 - protein, 16 aa, Artificial Sequence
  Description of Artificial Sequence Synthetic peptide
VRMNIQMPLEAPDYLE